(19) United States
(12) Patent Application Publication
(10) Pub. No.: US 2022/0125756 A1
(43) Pub. Date: Apr. 28, 2022

(54) THC-ALCOHOL APPARATUS AND METHOD OF MANUFACTURE THEREOF

(71) Applicants: Michael Roth, Scottsdale, AZ (US); Kevin Hazen, Flagstaff, AZ (US); Mason Cave, Glendale, AZ (US)

(72) Inventors: Michael Roth, Scottsdale, AZ (US); Kevin Hazen, Flagstaff, AZ (US); Mason Cave, Glendale, AZ (US)

(21) Appl. No.: 17/570,348

(22) Filed: Jan. 6, 2022

Related U.S. Application Data
(63) Continuation-in-part of application No. 17/569,176, filed on Jan. 5, 2022.
Application No. 17/569,176 is a continuation-in-part of application No. 17/553,512, filed on Dec. 16, 2021.
Application No. 17/553,512 is a continuation-in-part of application No. 17/553,448, filed on Dec. 16, 2021.
Application No. 17/553,448 is a continuation-in-part of application No. 17/550,866, filed on Dec. 14, 2021.
Application No. 17/550,866 is a continuation-in-part of application No. 17/547,183, filed on Dec. 9, 2021.
Application No. 17/547,183 is a continuation-in-part of application No. 17/124,396, filed on Dec. 16, 2020.
Application No. 17/124,396 is a continuation-in-part of application No. 17/120,138, filed on Dec. 12, 2020.
Application No. 17/120,138 is a continuation-in-part of application No. 17/115,640, filed on Dec. 8, 2020.
Application No. 17/115,640 is a continuation-in-part of application No. 17/111,366, filed on Dec. 3, 2020.
(60) Provisional application No. 63/105,261, filed on Oct. 24, 2020.
(60) Provisional application No. 63/278,811, filed on Nov. 12, 2021.
(60) Provisional application No. 63/277,366, filed on Nov. 9, 2021.
(60) Provisional application No. 63/183,202, filed on May 3, 2021.
(60) Provisional application No. 63/145,614, filed on Feb. 4, 2021.
(60) Provisional application No. 63/132,441, filed on Dec. 30, 2020.
(60) Provisional application No. 63/128,037, filed on Dec. 19, 2020.
(60) Provisional application No. 63/126,473, filed on Dec. 16, 2020.

(51) Int. Cl. A61K 31/352; A23L 33/105; A23L 2/52; A24B 15/167; A24D 1/18; A24D 1/00; A24B 15/40; A24B 15/32; A24B 15/28; B67C 3/02; B65D 17/00; B65D 17/28; B65D 23/14; B65D 1/02; B65D 85/72; A61K 36/185; A61K 9/00

(52) U.S. Cl. CPC A61K 31/352; A61K 2236/333; A23L 2/52; A24B 15/167; A24D 1/18; A24D 1/002; A24B 15/403; A24B 15/32; A24B 15/285; B67C 3/023; B65D 17/02; B65D 17/4014; B65D 23/14; B65D 1/02; B65D 85/72; A61K 36/185; A61K 9/0095; A23V 2002/00; B65D 2517/0032; A23L 33/105

(57) ABSTRACT

The invention comprises a method and apparatus for forming a Cannabis product, comprising the steps of: combining tetrahydrocannabinol and ethanol to form a THC-alcohol compound, the THC-alcohol compound comprising less than fifty percent water by mass and subjecting the THC-alcohol compound to a shear force in excess of 100 or 50,000 sec⁻¹. Optionally and preferably, formation of the Cannabis product results after successive steps of reducing viscosity of the tetrahydrocannabinol compound, such as by 2000+ cP and 20+ cP, or after reversing a first viscosity reducing step of 2000+ cP with a subsequent viscosity increase of 2000+ cP, such as in the formation of a THC containing drink, edible, vape pen solution, or preroll.

### CROSS-REFERENCES TO RELATED APPLICATIONS

This application is a continuation-in-part of U.S. patent application Ser. No. 17/569,176 filed Jan. 5, 2022, which is a continuation-in-part of U.S. patent application Ser. No. 17/553,512 filed Dec. 16, 2021, which is a continuation-in-part of U.S. patent application Ser. No. 17/553,448 filed Dec. 16, 2021, which is a continuation-in-part of U.S. patent application Ser. No. 17/550,866 filed Dec. 14, 2021, which is a continuation-in-part of U.S. patent application Ser. No. 17/547,183 filed Dec. 9, 2021, which:

is a continuation-in-part of U.S. patent application Ser. No. 17/124,396 filed Dec. 16, 2020, which is a continuation-in-part of U.S. patent application Ser. No. 17/120,138 filed Dec. 12, 2020, which is a continuation-in-part of U.S. patent application Ser. No. 17/115,640 filed Dec. 8, 2020, which is a continuation-in-part of U.S. patent application Ser. No. 17/111,366 filed Dec. 3, 2020, which claims the benefit of U.S. provisional patent application No. 63/105,261 filed October 24, 2020;

claims the benefit of U.S. provisional patent application No. 63/278,811 filed Nov. 12, 2021;

claims the benefit of U.S. provisional patent application No. 63/277,366 filed Nov. 9, 2021;

claims the benefit of U.S. provisional patent application No. 63/183,202 filed May 3, 2021;

claims the benefit of U.S. provisional patent application No. 63/145,614 filed February 4, 2021;

claims the benefit of U.S. provisional patent application No. 63/132,441 filed Dec. 30, 2020;

claims the benefit of U.S. provisional patent application No. 63/128,037 filed Dec. 19, 2020;

claims the benefit of U.S. provisional patent application No. 63/126,473 filed Dec. 16, 2020;

is a continuation-in-part of U.S. patent application Ser. No. 17/123,703 filed Dec. 16, 2020; and

is a continuation-in-part of U.S. patent application Ser. No. 17/120,135 filed Dec. 12, 2020,

all of which are incorporated herein in its entirety by this reference thereto.

### BACKGROUND OF THE INVENTION

### FIELD OF THE INVENTION

The present invention relates generally to a THC-alcohol product, such as in a beverage, edible, vape pen solution, or preroll.

### DISCUSSION OF THE RELATED ART

Food products shipped across state lines do not legally contain tetrahydrocannabinol and/or some nootropics.

Statement of the Problem

No system exists for generation of a THC-alcohol product subjected to shear forces.

### SUMMARY OF THE INVENTION

The invention comprises a method and apparatus for packaging a THC-alcohol product, such as in a beverage, edible, vape pen solution, or preroll.

### DESCRIPTION OF THE FIGURES

A more complete understanding of the present invention is derived by referring to the detailed description and claims when considered in connection with the Figures, wherein like reference numbers refer to similar items throughout the Figures.

FIG. 1 illustrates a two-location product preparation system;

FIG. 2 illustrates amendment additives;

FIG. 3 illustrates, in two-dimensions, a molecular structure of tetrahydro-cannabinol;

FIG. 4 illustrates a two-stage product preparation system;

FIG. 5 illustrates formation of a THC/additive stock solution;

FIG. 6 illustrates formation of an emulsion;

FIG. 7A illustrates methods of amendment of a starting product;

FIG. 7B further illustrates product amendment;

FIG. 8 illustrates a process of mixing, diluting, and filling a beverage holder;

FIG. 9 illustrates automated mass production;

FIG. 10 illustrates use of a binding agent;

FIG. 11 illustrates amendment adsorption and absorption;

FIG. 12 illustrates an injection process;

FIG. 13 illustrates control of an injection process;

FIG. 14 illustrates sequentially controlled injection;

FIG. 15 illustrates a multiple component formulation;

FIG. 16A illustrates a packaged formulation and FIG. 16B illustrates an amended formulation

FIG. 17 illustrates a THC amendment of an on-site produced formulation;

FIG. 18 illustrates adding THC to a beverage;

FIG. 19 illustrates amending product packaging;

FIG. 20 illustrates a beverage can;

FIG. 21 illustrates a can tab;

FIG. 22A illustrates a can safety lid, FIG. 22B illustrates a can safety lid in a closed orientation, and FIG. 22C illustrates a can safety lid in an open orientation;

FIG. 23A and FIG. 23B illustrate resealable lids with sliding and rotating openers, respectively;

FIG. 24 illustrates a rotated can tab;

FIG. 25 illustrates an adult can safety label;

FIG. 26 illustrates an adult labeled bottle;

FIG. 27 illustrates an emulsion formation capillary system;

FIG. 28 illustrates a shear inducement system;

FIG. 29 illustrates separated shear plates;

FIG. 30 illustrates shear plates;

FIG. 31 illustrates a THC room fogger;

FIG. 32A illustrates a room dispenser for THC and FIG. 32B, FIG. 32C, and FIG. 32D each illustrate THC dispensing rates;

FIG. 33 illustrates time delayed THC delivery;

FIG. 34 illustrates formation of a THC emulsion;

FIG. 35 illustrates use of multiple mixer types in formation of a THC emulsion;

FIG. 36 illustrates a high shear/force mixer system;

FIG. 37 illustrates repeated processing of a THC containing emulsion; and

FIG. 38 illustrates processing a THC containing emulsion with multiple processing types.

### DETAILED DESCRIPTION OF THE INVENTION

The invention comprises a method and apparatus for forming a Cannabis product, comprising the steps of: combining tetrahydrocannabinol and ethanol to form a THC-alcohol compound, the THC-alcohol compound comprising less than fifty percent water by mass and subjecting the THC-alcohol compound to a shear force in excess of 100 or 50,000 sec⁻¹. Optionally and preferably, formation of the Cannabis product results after successive steps of reducing viscosity of the tetrahydrocannabinol compound, such as by 2000+ cP and 20+ cP, or after reversing a first viscosity reducing step of 2000+ cP with a subsequent viscosity increase of 2000+ cP, such as in the formation of a THC containing drink, edible, vape pen solution, or preroll.

Herein, a food product refers to a solid food, a drink, and/or a beverage. While THC is optionally added to a food product, the THC and/or a form thereof is optionally added to a vape cartridge. Optionally, the food product refers to a first component of a subsequent food product, where the first component of the food product is packaged and labeled for sale, such as a syrup of a beverage.

Herein, for clarity of presentation and without loss of generality, tetrahydrocannabinol (THC) is used to represent any form of marijuana or Cannabis, such as CBD and/or CBG; THC is used to represent any molecule derived from THC, such as delta-8; and THC is used to represent any psychoactive substance found in the Cannabis plant. Stated again, where THC is used herein, optionally any of CBD, CBG, and/or delta-8 are optionally used with or without THC. And/or THC represents any mixture of 0-100% of each of THC, CBD, CBG, and delta-8.

Similarly, herein for clarity of presentation and without loss of generality, any nootropic, such as described herein, mushroom, and/or psychedelic, is optionally substituted from THC or Cannabis component and/or is used with THC and/or any Cannabis component, such as described supra.

The Cannabis plant, such as Cannabis sativa and/or Cannabis indica contains many useful constituents, such as CBD (cannabidiol), THC (Tetrahydrocannabinol), cannabinoids, terpenes, and/or other oils. Extraction and separation of constituents of the Cannabis plant and more generally any weed, herb, and/or spice is described herein. Generally, the use of colder extraction and separation techniques is preferred to retain more flavor, efficacy, terpenes, and flavonoids in end products. THC, CBD, and terpenes are further described infra.

THC

One component of the Cannabis plant is THC, which stands for tetrahydrocannabinol. THC is found in the marijuana plant. THC is one of at least 113 cannabinoids in Cannabis. Herein, the tetrahydrocannabinol and/or THC optionally refers to isomers of cannabinoid, tetrahydrocannabinol isomers, and/or (−)-trans-Δ⁹-tetrahydrocannabinol. Although the chemical formula for THC is (C₂₁H₃₀O₂), which describes many isomers, herein THC optionally and preferably refers to a principle psychoactive constituent of Cannabis, such as the Delta-9-THC isomer with chemical name (−)-trans-Δ⁹-tetrahydrocannabinol and/or a derivative thereof. Generally, THC is a lipid. THC is present in many forms, such as THCA, 2-COOH-THC, THC-COOH, and THCV. THC is soluble in hydrocarbons and alcohols. THC is widely regarded as effective in treatment of pain and many diseases.

Optionally, the THC used in any example herein is manufactured via distillation, extraction, and/or separation to a purity of greater than 25, 50, 75, 90, 92, 94, 96, or 98%. Optionally and preferably, the THC is distilled multiple times and/or extracted multiple times.

Optionally THC is reacted with a reagent, R, to form a THC derivative, such as a THC-R molecule, where the THC-R molecule retains and/or enhances psychoactive properties of THC, where the reagent, R, chemically aides dissolution, homogenization, solubility, and/or emulsification of the THC portion of the THC-R molecule in a body of a product, an aqueous based product, and/or a product containing greater than 10, 20, 30, 40, 50, 60, 70, 80, or 90 percent water and/or a hydrophilic substance, and/or where the reagent, R, chemically and/or physically decreases viscosity of a THC containing additive. For example, chemical reagent R and/or molecular sub-component R optionally and preferably has a hydrophilic end and an attachment end, where the attachment end bonds with THC and the hydrophilic end aids is dissolution in water or forming a suspension in water.

CBD

Another component of the Cannabis plant is CBD, which stands for cannabidiol. CBD is a second of the at least 113 cannabinoids in Cannabis plants. CBD is the second most prevalent of the active ingredients of Cannabis (marijuana). CBD is also derived from the hemp plant. Hemp, or industrial hemp, is a variety of the Cannabis plant, such as the Cannabis sativa plant species. CBD is present in many forms, such as: CBDA, CBC, CBN, CBG, and CBDV. CBD is also widely regarded as effective in the treatment of pain and many diseases.

Terpenes

Terpenes are aromatic compounds found in many plants, though many people commonly associate them with Cannabis because Cannabis plants contain high concentrations of them. Terpenes, which are aromatic compounds, are the source of characteristic scents of many plants, such as conifers, Cannabis, pine, lavender, and orange peel. Terpenes comprise compounds with the formula (C₅H₈)ₙ and are present in many forms, currently numbered at more than 30,000 forms. Generally, terpenes are unsaturated hydrocarbons. Terpenes are further classified by the number of carbons: monoterpenes (C₁₀), sesquiterpenes (C₁₅), diterpenes (C₂₀), C₂₅, C₃₀, C₃₅, C₄₀. Strictly speaking all monoterpenes have the same chemical formula C₁₀H₁₆. Similarly all sesquiterpenes and diterpenes are respectively C₁₅H₂₄ and C₂₀H₃₂. The structural diversity of mono-, sesqui-, and diterpenes is a consequence of isomerism. Herein, a terpene refers to any form and/or isomer of the above described unsaturated hydrocarbons. Terpenes can be visualized as the result of linking isoprene (C₅H₈) units “head to tail” to form chains and rings. A few terpenes are linked “tail to tail”, and larger branched terpenes may be linked “tail to mid”.

Terpenes have chemical properties that may be used in extraction and/or separation processes, such as described herein. Terpenes are colorless, although impure samples are often yellow. Terpene boiling points scale with molecular size. Boiling points of terpenes, sesquiterpenes, and diterpenes are respectively 110±20, 160±20, and 220±20° C. Terpenes are non-polar and are considered to be insoluble in water. Due to the low specific gravity, density, of terpenes, terpenes float on water. As terpenes are hydrocarbons, they are highly flammable.

Terpenoids (mono-, sesqui-, di-, etc.) have similar physical properties to terpenes; however, terpenoids tend to be more polar and hence slightly more soluble in water and somewhat less volatile than their terpene analogues. Highly polar derivative of terpenoids are the glycosides, which are linked to sugars, which are water-soluble solids. Terpenoids are tactilely light oils, which are considerably less viscous than familiar vegetable oils, such as corn oil with a viscosity of 28 cP. Generally terpenoids have a viscosity ranging from 1 cP, similar to water, to 6 cP. s

Terpenes in Cannabis

In terms of the Cannabis plant, terpenes are the natural compounds in the flower or bud of the Cannabis plant. Terpenoids, meanwhile, are terpenes that have been chemically altered. The process of drying and curing a Cannabis flower leads to the formation of terpenoids from their respective terpene. In Cannabis, the terpenes are secreted in the same glands that produce cannabinoids like THC and CBD. In Cannabis, terpenes are aromatic oils that color Cannabis varieties with distinctive flavors like citrus, berry, mint, and pine.

While there are a wide variability of terpenoid compositions, Cannabis strains are dominated by six terpenoids listed here in order of most common to least common: Myrcene (˜43%), β Caryophyllene group (˜23%), Limonene group (˜16%), Terpinolene (˜10%), α Pinene (˜6.5%), and the β-ocimene outlier group (<1%).

While the dominant terpenes are the myrcene (˜43%), caryophyllene, limonene group (˜16%), terpinolene (˜10%), pinene, and ocimene terpenes or terpene groups, many less dominant terpenes are present in Cannabis, such as: 3-carene; camphene; caryophyllene oxide; fenchol; humulene; linalool; alpha-phellandrene (α-phellandrene); beta-pinene (β-pinene); alpha-terpinene (α-terpinene); gamma-terpinene (γ-terpinene); and terpineol. Terpenes in a typical terpene profile analysis include one or more of: isopulegol, menthol, linalool, caryophyllene oxide, guaiol, Sabinene, p-cymene, Camphene, eucalyptol, geraniol, terpinolene, alpha-bisabolol, alpha-pinene, alpha-terpinene, beta-caryophyllene, beta-pinene, delta-3-careen, L-fenchone, beta-myrcene, alpha-phellandrene, alpha-ocimene, D-limonene, cis-beta-ocimene, gamma-terpinene, alpha-humulene, cis-nerolidol, and trans-nerolidol. Herein, terpenes optionally refer to both terpenes and terpenoids.

The boiling point and densities for common terpenes found in strains of Cannabis that are associated with THC and CBD are presented in Table 1. The boiling point and/or density is optionally and preferably used in the extraction and/or separation processes described herein.

TABLE 1
Common Terpenes Associated with THC and CBD

| | | Boiling | Boiling | |
|---|---|---|---|---|
| | | Point | Point | Density |
| THC Terpenes | CBD Terpenes | (° F.) | (° C.) | (g/cm³) |
| Myrcene | Myrcene | 332 | 167 | 0.794 |
| Caryophyllene | Caryophyllene | 266 | 130 | 0.905 |
| Limonene | Limonene | 348 | 176 | 0.842 |
| Terpinolene | | 366 | 186 | 0.861 |
| Pinene | Pinene | 311 | 155 | 0.858 |
| Ocimene | | 122 | 50 | 0.800 |
| Linalool | Linalool | 388 | 198 | 0.858 |
| Humulene | Humulene | 222 | 106 | 0.886 |
| | Guaiol | 197 | 92 | 0.961 |
| | Eucalyptol | 342 | 172 | 0.922 |

Chemically, terpenes are organic compounds made up of isoprene molecules, also known as isoprene units H₂CC(Me)CHCH₂, a five carbon atom building block containing a double bond. While the terms, terpenes and terpenoids are often used interchangeably, terpenoids are a class of phytochemicals derived from terpenes which have additional functional groups, often including oxygen or cyclic structures. Terpenes have two or more isoprene units as their common structural feature and are classified according to the number of contained isoprene units. Many terpenes are extremely volatile, are readily oxidized, and as such, are readily damaged/altered in extraction and separation processes. Thus, keeping temperatures down in an extraction process, such as below 160, 150, 140, 130, 120, 110, 100, 90, or 80° F. is beneficial. Further, lowering temperature from room temperature to less than 75, 70, 65, 60, 50, or 40° F. is beneficial. The fewer the number of bonds and/or isoprene units the easier the terpene volatilizes or boils off. Common classifications of terpenes are monoterpenes, sesquiterpenes, and diterpenes. Monoterpenes contain two isoprene units. These are the most abundant type of terpenoids in Cannabis, and they contain all but one of the “primary” terpenes. Common monoterpenes include limonene, myrcene, pinene, ocimene, terpinolene, and linalool. Sesquiterpenes contain three isoprene units, where “sesqui-” means “one and a half”. Sesquiterpenes common in Cannabis include β-caryophyllene, α-bisabolol, humulene, valencene, nerolidol, caryophyllene oxide, and guaiol. Diterpenes contain four isoprene units. Phytol is a common diterpene and is a breakdown product of chlorophyll and tocopherol.

Herein, for clarity of presentation and without loss of generality, tetrahydrocannabinol (THC) is used to illustrate a component that is legal in a second location, such as a licensed THC facility, that is not legal in a first location, such as a manufacturing facility. More generally, many regulated components, formulations, and/or chemicals are legally packaged in a second location where the many regulated components, formulations, and/or chemical may not be legally packed at the first location or shipped from the first location to the second location.

Herein, for clarity of presentation and without loss of generality, a processed cheese sauce is illustrative of a manufactured formulation prepared at a first location, such as a main manufacturing facility that is amended, such as with the addition of tetrahydrocannabinol, at a second facility. Other products that are optionally manufactured at one facility and amended at a second facility include, but are not limited to: whipped cream, icing, cookie dough, or pancakes, where any of the products are optionally delivered from a pressurized canister. More generally, any food product prepared and packaged for sale at a first location is optionally amended, to form an amended food product, at a second location, such as a sweet, such as a chocolate, a savory item, such as a cheese puff, and/or a beverage, such as a soda.

Herein, an original food product is optionally packaged for sale in a pressurized container, such as a sprayable cheese product. For clarity of presentation and without loss of generality, examples are provided that amend the originally packaged sprayable cheese product to form an amended sprayable cheese product, such as containing THC. However, generally any originally packaged food/drink product is optionally amended, such as described herein, to form an amended food/drink product, such as an amended gummy, chip, pretzel, snack, candy, baked good, bagged food product, boxed food product, beverage, canned drink, and/or bottled drink.

Herein, for clarity of presentation and without loss of generality, a pressurized device includes, but is not limited to, a pressurized canister, an aerosol canister, a bag-in-can type canister, and/or a piston barrier system, which includes a canister with a product on a dispensing side of the piston and a pressurized gas on the opposite side of the piston. Typically, in an aerosol canister, the propellant is at least partially delivered with the product. Typically, dispensing product from the piston barrier system results in little to no co-dispensing of the pressurized gas until after the product is substantially dispensed, such as greater than 95% of the product has been dispensed.

Herein, an x/y-plane is perpendicular to a z-axis aligned with gravity.

Multiple Location Product Manufacturing

Generally, a food product is made at a first location, shipped, sold and/or is consumed/used at a second location. Typically, the first location is a manufacturing facility, such as in a first state and the sale location and/or point of consumption/use is at a retail facility or residence, such as in a second state. Some formulations do not ship well and/or are best shipped without certain elements in the formulation, such as a component legal in one location and illegal in a second location. For instance, THC placed into an original food product is not currently legally shipped across state lines in the United States of America. Hence, as described herein, an original food product is optionally sequentially: produced for sale in a first state, shipped across a state line into a state where THC in the food product is legal, amended with THC, and subsequently distributed for sale as an amended product.

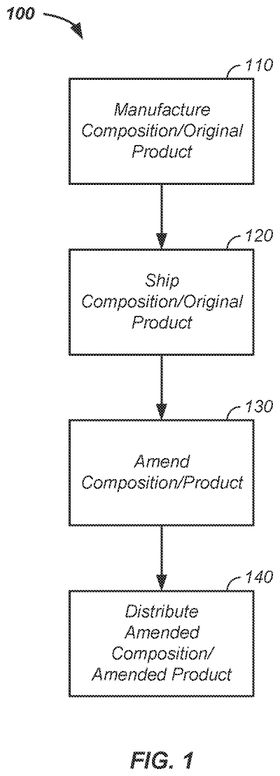

Referring now to FIG. 1, a multiple location product manufacturing system 100 is illustrated. Notably, the multiple location product manufacturing system 100 does not refer to a complete product being manufactured at a first location and the same complete project being manufactured at a second location. Rather, the multiple location product manufacturing system 100 refers to a sequence of processes described herein. In a first process, manufacture of a composition and/or a product 110 is performed at a first location to form an original product 112, such as a product for sale to an end consumer. Subsequently, in a second process, the composition and/or the original product 112 is shipped 120, such as across a state line, from a first legal jurisdiction to a second legal jurisdiction, from a location where THC is illegal to a location where THC is legal, to a regulated facility, and/or to a THC certified facility. After shipment, a third process of amending 130 the composition and/or amending the original product 112 is performed, such as an addition of THC to the original product 112 to form an amended product 114. The amendment process 130 optionally includes additional steps, such as the addition of a THC emulsion and/or opening of a pressure seal and/or inserting at least one composition element into a pressurized environment of the manufactured and shipped composition and/or the manufactured and shipped product. In a fourth process, the amended composition and/or the amended product, is shipped and/or distributed 140, such as for sale.

### Example I

Still referring to FIG. 1, in a first example of the multiple location product manufacturing system 100, a process of amending the composition/product with a process of amending the composition/product with at least one added constituent is further described. For clarity of presentation and without loss of generality, the added component in examples herein is THC. Optionally, the added component includes one or more of: THC, a hallucinogen, a psychedelic, a dissociate, a deliriant, and/or a designer drug, where the designer drug contains a structural and/or a functional analog of a controlled substance that has been designed to mimic the pharmacological effects of the original drug while at the same time avoiding being classified as illegal. Optionally, the added component includes a chemical, a class of chemicals, a molecule, a class of molecules, a compound, and/or a composition illegal in a first geographic zone, such as at a first manufacturing facility, and legal in a second geographic zone, such as at a product amendment facility. Optionally, the added component includes a mushroom and/or a nootropic, such as a vitamin, limonella, an extract of Eustis Limequat, an extract of a fruit, a portion of a peel of a fruit, a zest, a terpene, and/or niacin. In a first case, the manufactured and shipped product, such as the original product, is in a pressurized environment, such as in an aerosol canister or is packaged in a product side of a piston barrier in a container of a piston barrier system. In this first case, at the second location, the pressure barrier is optionally opened, such as to a higher pressure environment containing THC, THC is added to the aerosol canister which flows into the container as a result of the higher pressure in the THC additive environment, and the container is then resealed in preparation for distribution and/or sale. In a second case, the original product is shipped ready for sale in a first package. The package is opened, the contents are amended, and the amended contents are distributed for sale, such as in the original packaging or in new packaging.

Amendment Additives

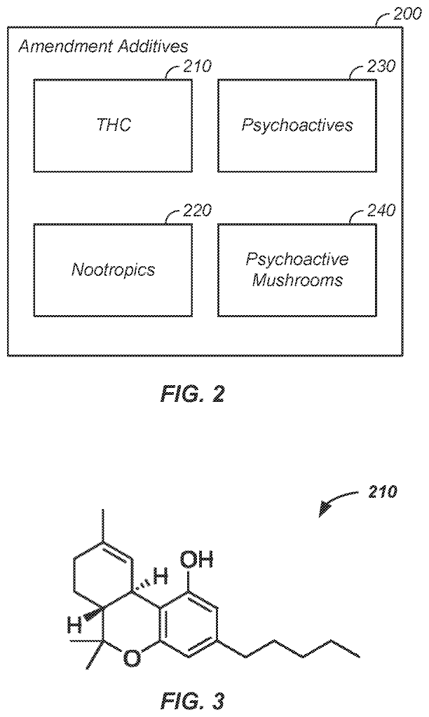

Referring still to FIG. 1 and referring now to FIGS. 2 and 3, generally the multiple location product manufacturing system 100 is used to amend the original product 112 with an additive 200. Optionally, the packaging of the original product 112 is amended, as further described infra. Herein, for clarity of presentation and without loss of generality, tetrahydrocannabinol 210 is used as an example of the additive 200. However, any additive 200 is optionally used in the step of amending the composition/product 130. For example, still referring to FIG. 2, the additive 200 is optionally a nootropic 320, such as any agent that makes you feel emotionally happier, stronger, and/or better. Herein, a nootropic is optionally any natural product, molecule, formulation, or mixture that is currently, as of the year 2020, legal in one or more states in the United States of America and is currently illegal in one or more other states America. A nootropic 220 is not necessarily science backed; however, many nootropics have known effects on the body. For instance, the additive 200, is optionally a psychoactive 230, such as a molecule, substance, or mixture, that affects the nervous system resulting in alterations in perception, mood, consciousness, cognition, or behavior. For example, psychoactive mushrooms 240 are currently legal in some states, such as Colorado. Additional examples of nootropics 220 include a depressant, a stimulant, MDMA, an anxiolytic, nicotine, a barbiturate, and/or a hallucinogen. Optionally, a nootropic is legal in all states. For instance, niacin is an example of a nootropic and ingestion of niacin results in a flush or warm feeling. Caffeine, an example of a stimulant nootropic, is another example of a nootropic, which is a neural system stimulant. Nootropics also include: melatonin, Gaba, 5-htp, L-theanine, Bacopa monnieri, Rhodiola rosea, Panax ginseng, citicoline, L-Tyrosine, alpha GPC, Huperzine A, Bacopa Monnieri, phosphatidylserine, N-Acetyl-L-Tyrosine, mushroom, vitamin C in combination with mushrooms as vitamin-C increases bioavailability of mushrooms, Valerian root, omega 3s, Lion mane mushroom, Cordyceps militaris and sinensis, Reishi (Ganoderma lucidum), Chocolate (cocoa powder), caffeine, MCT oils, magnesium, Ashwagandha Root Extract, vitamin-D, carotenoids, algae amino acids, jellyfish extract, and/or terpenes. In addition, each of tryptophan, 5-htp, SAMe (S-adenosyl-L-methionine), St. John's wort, and probiotics increase serotonin production and/or function to modulate the serotonin pathway. One combination of nootropics is Gaba, Myrcene, and THC. Additional optional nootropics include:
1. 5-HTP for serotonin regulation;
2. Acetyl L-Carnitine to protect and power brain cells;
3. Alpha Lipoic Acid, an antioxidant for blood-brain barrier protection;
4. Alpha-GPC a phospholipid choline to support brain structure;
5. Aniracetam, a synthetic Russian racetam, for mood enhancement;
6. Apoaequorin, jellyfish protein for neuroprotective activity;
7. Artichoke supplies luteolin, for mental performance;
8. Ashwagandha for mental energy;
9. Astaxanthin a potent antioxidant found in algae and seafood;
10. Bacopa monnieri for improved retention of knowledge;
11. Caffeine, not a true nootropic, but a stimulant;
12. California poppy interacts with GABA and HTP receptors for anxiety-soothing;
13. Cat's Claw for neuroprotective activity;
14. Catuaba, a traditional Brazilian bark herb, for neuroprotection;
15. CBD, a Cannabis derived compound, to ease anxiety;
16. Celastrus paniculatus, woody shrub seeds, for brain health;
17. Centrophenoxine, a synthetic smart drug related to DMAE for enhanced cerebral vascular function;
18. Citicoline, a choline source, for brain energy and mood enhancement;
19. Clitoria ternatea, a traditional Asian herb, for memory enhancement;
20. Coluracetam, called MKC-231, a synthetic racetams for help with brain degeneration;
21. Convolvulus pluricaulis, an Indian tonic herb, aids learning;
22. CoQ10 helps powering brain cell mitochondria;
23. Creatine for charging muscles;
24. Choline for maintaining healthy brain structure;
25. DHA for early brain development;
26. DMAE, found in sardines, for mood enhancement;
27. Forskolin, active ingredients of Coleus forskohlii, for regulation of cell-to-cell communication;
28. GABA, an inhibitory amino acid, settles nerves producing relaxation and a pleasant mood;
29. Ginkgo biloba for enhancing brain circulation;
30. Ginseng for countering stress;
31. Gotu Kola for blood vessel support;
32. Guarana to boost physical endurance;
33. Huperzine-A, a synthetic alkaloid, to help with degenerative brain concerns;
34. Kanna (Sceletium tortuosum) for help with mental performance;
35. Kava Kava, a psychoactive root to ease anxiety;
36. Kratom to promote feelings of calmness and positivity;
37. L-Glutamine for healthy cognition;
38. L-Phenylalanine for mood balance;
39. L-Theanine to promote wakeful relaxation;
40. L-Tryptophan for calm/relaxed moods;
41. Lecithin supplies for optimizing brain cell healthy structure;
42. Lemon Balm to promote calmness;
43. Lion's Mane Mushroom for brain plasticity;
44. Magnolia for relaxation;
45. MCT Oil for brain energy;
46. NADH for ATP energy production;
47. Nefiracetam, a synthetic racetam smart drug for memory;
48. Nicotine, not a nootropic, but has shown nootropic effects in the realm of brainpower;
49. Noopept, patented racetam;
50. Oatstraw for relaxed alertness;
51. Oxiracetam for focus;
52. Passionflower for relaxation;
53. Phenibut for mood;
54. Phenylpiracetam for cognitive function;
55. Phosphatidylcholine for brain regeneration;
56. Phosphatidylserine helps build, power, and protect brain cells;
57. Picamilon for anxiety;
58. Pine Bark Extract for attention;
59. Piracetam for stimulating, mood balancing effects;
60. Psychobiotics as beneficial flora in the GI tract can influence mood and cognitive function;
61. Pramiracetam for memory formation;
62. Pterostilbene for resistance to aging;
63. PQQ for the production of energy within brain cells;
64. Resveratrol, a red wine antioxidant for protecting brain cells against free radicals and inflammation;
65. Rhodiola rosea for mental energy and physical endurance;
66. Rosemary for age-related cognitive support;
67. SAMe for mood balance;
68. Schizandrol-A for anti-stress;
69. St. John's Wort for helping with depression;
70. Sulbutiamine for brain health;
71. Taurine for nerve-calming effects;
72. Theobromine for stimulating properties;
73. Turmeric for cognitive health;
74. Tyrosine for mental performance in distracting, multitasking settings;
75. Uridine for brain regeneration;
76. Valerian for sustaining GABA levels to promote relaxation;
77. Vinpocetine, a synthetic form of periwinkle, for brain circulation;
78. Vitamin B1 (Thiamine) to help brain chemicals to function properly;
79. Vitamin B3 (Niacin) for healthy brain function;
80. Vitamin B5 (Pantothenic Acid) for help with attention-related issues;
81. Vitamin B6—for nerve sheathing and blood vessel flexibility;
82. Vitamin B8—also called inositol, for brain cell membrane synthesis;
83. Vitamin B9 for regulating homocysteine and cerebrovascular health;
84. Vitamin B12 for brain energy;
85. Yerba Mate for focus-enhancing support;
86. Cannabigerol (CBG) and/or a decarboxylated form of cannabigerolic acid, which is optionally derived/extracted from Cannabis and/or is synthetically produced; and
87. Vanilla.

Some nootropics are illegal in one or more states and are legal in one or more different states in the United States of America, such as Psilocybe, psilocin, baeocystin, Cubensis, and Panaeolus (Copelandia).

Additive: Alcohol Flavoring

Another optional additive to any of the THC compounds, formulations, emulsions, and/or tinctures described herein is an alcohol flavoring, which is used with or without alcohol, such as in a beverage. Generally, the optional additive is any chemical, plant extract, plant derivative, derivative from a plant part, and/or flavoring that leads to a taste and/or feel of a traditional alcoholic beverage. For example, gin flavoring is derived from berries, such juniper berries, wild plum, a tart plum, a sour plum, a dark purple fruit, blackthorn, sloe, a wild American plum, and/or an astringent fruit, and/or is derived from juniper and/or evergreen, where either are used with or without angelica spice. Similarly, amaretto flavoring is derived from either of and preferably a blend of extracts from almonds and apricot kernels. Similarly, tequila flavoring is optionally provided with an ester, such as ethyl hexanoate; an alcohol, such as 2-phenyl ethanol and/or isoamyl alcohol; a ketone or an essential oil, such as a damascenone and/or beta-damascenone; an Agave nectar, such as a cooked piňa of Agave; an acetaldehyde, such as isovalderaldehyde that yields a sweet, cocoa, and chocolate-like flavor; a 2- and/or 3-methylbutanal, which yields a malty flavor; and/or a phenolic, such as vanillin and/or syringaldehydes that yields a strong, fruity, and/or herbal aroma; and/or eugenol for a spicy flavor. Tequila and other alcoholic beverages often involve heating, which results in Maillard browning reactions, yielding furans from the thermal degradation of sugar, which are optionally added to the THC product. For example, 2-furaldehyde and/or 5-methylfuraldehyde are optionally and preferably added as flavorings to a tequila like beverage with or without alcohol. Smoky flavorings include guaiacol and again beta-damascenone. Optionally and preferably, the container containing the THC is labeled as also containing a flavoring mimicking an alcoholic drink, such as “tequila flavoring”, gin flavoring, or the like.

Referring now to FIG. 3, tetrahydrocannabinol 210 is illustrated. Tetrahydrocannabinol (THC) is one of at least 113 cannabinoids identified in cannabis. Herein, the tetrahydrocannabinol and/or THC optionally refers to isomers of cannabinoid, tetrahydrocannabinol isomers, and/or (−)-trans-Δ⁹-tetrahydrocannabinol. Tetrahydrocannabinol is the principal psychoactive constituent of cannabis. Optionally THC is reacted with a reagent, R, to form a THC derivative, such as a THC-R molecule, where the THC-R molecule retains and/or enhances psychoactive properties of THC, where the reagent, R, chemically aides dissolution, homogenization, solubility, and/or emulsification of the THC portion of the THC-R molecule in a body of the product, an aqueous based product, and/or a product containing greater than 10, 20, 30, 40, 50, 60, 70, 80, or 90 percent water and/or a hydrophilic substance, and/or where the reagent, R, chemically and/or physically decreases viscosity of a THC containing additive, which aids in homogenization, distribution, and/or mixing of the THC into a viscous product, such as a cheese product and/or a cookie dough. For example, chemical reagent R and molecular sub-component R optionally and preferably has a hydrophilic end and an attachment end, where the attachment end bonds with THC and the hydrophilic end aid is dissolution in water or forming a suspension in water.

Optionally, the THC 210 used in any example herein is manufacture via distillation or extraction to a purity of greater than 25, 50, 75, 90, 92, 94, or 96%. Optionally and preferably, the THC is distilled multiple times and/or extracted multiple times, which reduces changes the THC from a mowed lawn flavor to flavorless, such as after three sequential distillations or an equivalent laboratory grade distillation.

Still referring to FIGS. 1-3, the multiple location product manufacturing system 100 used to amend the original product 112 with an additive 200 to form an amended product 114 is further described. For clarity of presentation and without loss of generality, two examples are provided of amending the original product 112 to form an amended product 114.

### Example I

In a first example, an original product 112, such as a chocolate snack is manufactured where adding THC at greater than two milligrams per serving is illegal; the original product 112 is shipped to a second location where THC is legal; and THC is added to the chocolate to form the amended product 114.

### Example II

In a second example: (1) an original product 112, such as a hot chocolate mix, a coffee additive, or a whipped cream is manufactured and is optionally labeled for sale to an end customer; (2) the original product 112 is shipped from a first location where addition of the additive 200, such as at an effective dose of the additive 200 to produce a psychoactive event, is illegal to a second location where addition of the additive 200, at the effective dose, is legal; and (3) the original product 112 is amended with an effective dose of the additive to form the amended product 114; and (4) optionally the amended product is distributed for sale in locations where the additive 200 is legal and/or shipment is legal.

Multiple Location Product Preparation

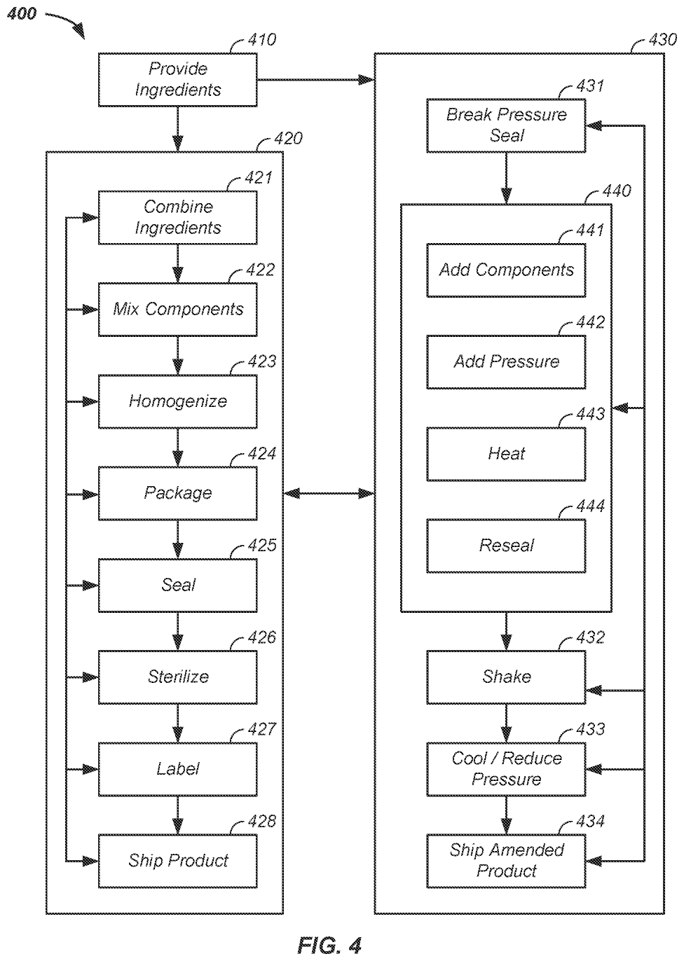

Referring now to FIG. 4, a multiple location product preparation system 400 is described. Optionally and preferably, the multiple location product preparation system 400 is implemented as a portion of the multiple location product manufacturing system 100; however, any and/or all of the steps of the multiple location product preparation system 400 are optionally performed within a single location, such as a THC licensed manufacturing location. Generally, the multiple location product preparation system 400 includes a first location 420 and a second location 430, where zero, one, or more steps of manufacturing a product are performed in each of the first location 420 and/or the second location 430. For example, a described heating step, pressurizing step, shaking step, time passing step, resealing step, and/or sterilization step are optionally performed more than once, such as at the first location and subsequently at the second location. In another example, a step described herein for clarity of presentation and without loss of generality at the first location 420 is optionally performed only in the second location 430 without performing the step in the first location 420. The multiple location product preparation system 400 is further described in the non-limiting examples herein.

### Example I

Still referring to FIG. 4, a first example of the multiple location product preparation system 400 is provided. In this example, a product formulation includes at least a set of constituents along with optional steps to assemble the ingredients to form the product.

In this first example, in a first step, a first portion of the ingredients is provided 410 to the first location 420, such as a first manufacturing location. Optionally and preferably, a second portion of the ingredients is provided to the second location 430, such as a second manufacturing location. In this example, the provided first ingredients 410 are combined 421, mixed 422, homogenized 423 and/or emulsified, packaged, 424, sealed 425, sterilized 426, labeled 447, and/or shipped 428. Optionally and preferably, a first set of sub-components of the provided ingredients are combined using one or more of the steps described herein into a first sub-mixture and a second set of sub-components of the provided ingredients are combined using one or more of the steps described herein into a second sub-mixture, where the number of sub-mixtures is any integer n, where n is a positive integer of greater than 1, 2, 3, 4, 5, or more. For instance, the first set of sub-components are combined and formed into an emulsion, such as with a homogenizer, which results in the first mixture. Subsequently, the second mixture is combined with the emulsified first mixture. The emulsification first process aids in forming a uniform distribution of each component in the resulting product, aids in dissolving an oil into an aqueous mix or vice-versa, and/or aids in homogenization of ingredients added to the already formed emulsion.

Still referring to FIG. 4, in one optional and preferred embodiment, the product formed at the first location is ready for distribution and sale. Said again, without any additional step at the second manufacturing location, the product is ready for sale, such as in a retail store to an end customer, an end user, and/or an end consumer. For instance, whipped cream, a spreadable/sprayable cheese, a cookie dough, an icing, a snack, a sweet, and/or a savory item, is prepared and is ready for sale from the first location, such as at a retail facility to a person who will consume the originally manufactured product. Optionally, the original product is amended at the second location, such as by the addition of THC at a THC licensed manufacturing facility. In another embodiment, an incomplete product is formed at the first location that is not fully ready for sale to an end consumer, such as a syrup used in a soda fountain machine. In this case, the incomplete product is amended and/or finalized at the second location, such as at a THC licensed manufacturing facility.

In this first example, still referring to FIG. 4, in a second step the product and/or the incomplete product formed at the first manufacturing location is subsequently shipped 428 to a second location 430. For clarity of presentation and without loss of generality, several cases of shipping from the first location 420 to the second location 430 are provided in Table 2. For instance, the product and/or the incomplete product is optionally manufactured at a first location in a state, such as Arizona, where addition of THC to a food product is illegal, then the product is shipped to a THC licensed manufacturing facility in Arizona where manufacturing of the incomplete product is finalized and/or the product is amended, such as through addition of one of more constituents, such as an optional THC component. Similarly, the product and/or the incomplete product is optionally manufactured in Utah where addition of THC to a food product is illegal, then the product is shipped to a THC licensed manufacturing facility in Arizona where manufacturing of the incomplete product is finalized and/or the product is amended, such as through addition of one of more constituents that optionally includes THC.

TABLE 2
Shipping

| First Location | Second Location |
|---|---|
| in state at non-THC licensed | in same state at THC licensed |
| manufacturing facility | manufacturing facility |
| first state non-THC licensed | second state THC licensed |
| manufacturing facility | manufacturing facility |
| first government zone/region/area | second government zone/region/area |
| prohibiting THC containing | allowing THC containing |
| product production | product production |

In this first example, still referring to FIG. 4, in a third step the incomplete product and/or original product 112 manufactured at the first location 420 is optionally completed and/or amended at the second location 430 to form the amended product 114. For instance, in a sub-case where the incomplete product and/or the product manufactured at the first facility is contained in a pressurized package, such as at greater than one atmosphere, an optional and preferred step is breaking the pressure seal 431. Subsequently or for a non-finalized product or product that is not contained in a pressurized packed, the product is amended in an amendment step 440. Herein, for clarity of presentation, the non-finalized product and the product shipped from the first location 420 are both referred to as original products 112, which are subsequently operated on, finalized, and/or amended at the second location 430 to form the final product(s) 114. In the amendment step 440, optionally and preferably components are added 441. For example, THC is amended into the product, such as further described infra. Optionally, the product is also pressurized, repressurized, and/or pressurized to a higher pressure 442; heated 443; and/or resealed 444. Notably, any other manufacturing step described herein or commonly performed is optionally additionally performed as part of the amendment step 440, such as mixing 422, homogenizing 423, sterilizing 424, and/or re-sealing 424. Notably, after and/or as part of the amendment step 440, one or more additional steps optionally occur, such as shaking the current product 432, heating the product, such as to alter a viscosity of one or more constituents of the product, cooling the product, reducing pressure in the container 433, and/or heating the product, such as in a water bath sterilization step. In one case, an ultrasonic mixer is used to mix in the amended constituents, such as THC, into the original product to form the amended product. Similarly, in a second case, an ultrasonic resonator, which is distinct from an ultrasonic mixer, is used to homogenize a product amended with a supplemental component, such as THC, to form the amended product. The ultrasonic resonator uses ultrasonic waves that resonate in phase with a natural frequency of a mixable object. For instance, a tube of cheese will have a resonant frequency and the ultrasonic resonator applies that resonant frequency to the tube of cheese to mix the tube of cheese. Further, the aforementioned steps of labeling 447 optionally occurs at any time at the second location 430. After the process of adding components 441 to the product, the product is optionally referred to as an amended product, a final product, or simply the product. The amended product is then optionally and preferably distributed/shipped 434 to a retail facility for sale, such as a marijuana dispensary.

Centipoise (cP) is a measurement of viscosity, which is sometimes written as cps. While pure THC is viscous, the viscosity of THC dramatically decreases with temperature. For example, viscosities of two pure THC samples as a function of temperature are provided in Table 3. As provided in Table 3, the viscosity of pure THC decrease from a very thick almost non-fluid paste at room temperature to a less viscous flowing honey like substance at 40-60° C., with viscosities in the 500 to 3000 cP range, to a much more fluid substance at 65-70° C., with viscosities in the 100 to 400 cP range.

TABLE 3
THC viscosity

| Temperature | Sample 1 | Sample 2 |
|---|---|---|
| (° C.) | Viscosity (cP) | Viscosity (cP) |
| 40 | 8060 | 5666 |
| 45 | 3595 | 2701 |
| 50 | 1794 | 1385 |
| 55 | 952 | 760 |
| 60 | 537 | 439 |
| 65 | 319 | 268 |
| 70 | 199 | 170 |

The viscosity of THC is optionally reduced by mixing it with ethanol, which reduces the viscosity from 5000+ cP to less than 500, 250, 100, or 50 cP, such as by mixing in ethanol to the THC at a ratio of greater than 1:100, 1:50, 1:10, 1:2, 1:1, or 2:1 ethanol:THC by mass or volume. The viscosity reduction of mixing THC with ethanol is optionally and preferably enhanced by increasing the temperature of the THC-ethanol compound. The viscosity of THC is optionally similarly reduced by mixing the THC with an oil, fat, a triglyceride, and/or butter.

Once the viscosity of the THC or a THC-ethanol compound is reduced to a flowable fluid, such as with a viscosity of less than 10,000, 5000, 1000, 500, 200, 100, or 50 cP, the THC and/or the THC-ethanol compound is optionally subjected to a shear force, such as by passing through a sprayer. Optionally and preferably, the sprayer applies a shear force of greater than 1, 10, 50, 100, 1000, 10,000, 100,000, or 1,000,000 sec⁻¹ to the THC and/or the THC-ethanol compound, such as in an spraying application step, injection application step, mixing step, brushing step, immersion step, and/or emulsification step as described herein. Herein, spraying with air optionally results in shear forces in the 1 to 10,000 sec⁻¹ range and spraying with an airless sprayer and/or passing through a high shear mixer results in shear forces in the 10,000+ sec⁻¹ range. Optionally and preferably, in a first step the viscosity of THC is reduced, such as from above 5,000 cP to less than 5,000, 2500, 1,000, 500, or 300 cP by heating and/or mixing with a solvent, such as a fat, oil, and/or ethanol. In a second step, the viscosity of the resulting THC compound is optionally further reduced to less than 300, 250, 200, 100, 10, 5, or 2 cP by mixing the THC compound with beverage components, such as water, a sweetener, and/or a flavoring. Optionally, in the second step, the THC compound is mixed with and/or is applied to an edible where the viscosity of the final product is increased to greater than the end viscosity of the second step and/or the viscosity of the final product is increased by cooling the product, such as to room temperature. In this case, the final edible product may have a viscosity of greater than 300, 500, 1000, 2500, 5,000, or 10,000 cP.

Product Amendment

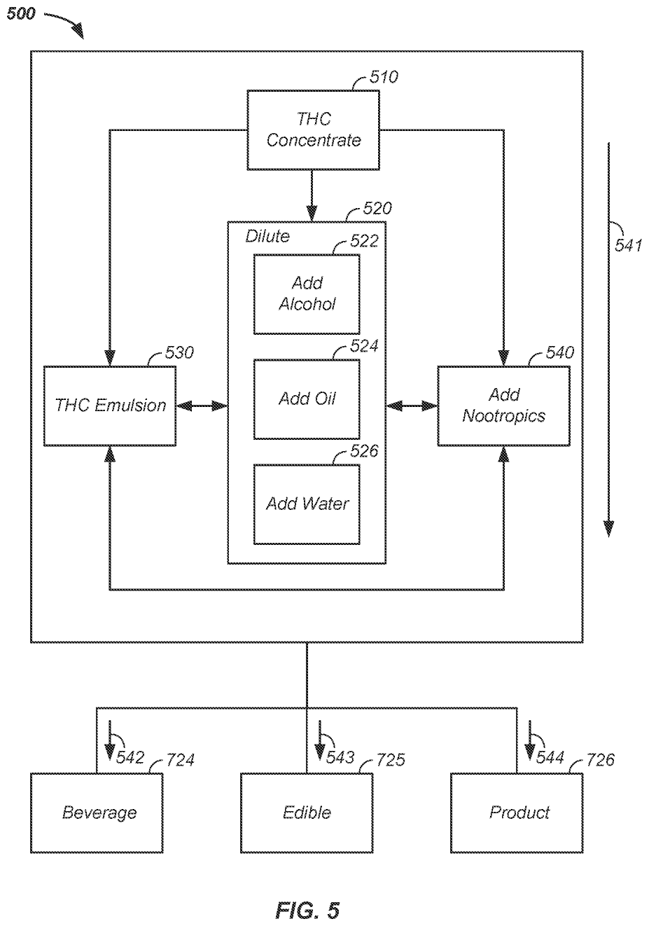

Referring now to FIGS. 5-15, the step of amending the composition/product 130 is further described. Generally, a raw ingredient, such as THC 210 is manipulated to: (1) form a stock solution, such as a THC stock solution 500 additive, such as a formulation that is diluted, compounded, homogenized, and/or an emulsified, where the generated stock additive has chemical and/or physical properties that facilitate a subsequent step of being added into and/or onto the original product 112 and (2) a given original product 112 is treated with the stock additive, which is a formulation of the additive 200, to form the final product 114. Typically, the stock additive is injected into, dropped onto, sprayed on, or mixed into the original product 112. The amendment process if further described infra.

Preparation of Stock Additive

Referring now to FIG. 5, the process of forming the stock additive 500 is further described. Generally, the stock additive 500 is of any additive, with or without THC. However, again for clarity of presentation and without loss of generality, formation of a THC stock is used to described the process of forming any stock additive. The relatively pure form of THC is also herein referred to as a THC concentrate. Herein, the THC concentrate is processed by a manufacturer into a relatively pure form, such as greater than 50, 60, 70, 80, 85, 90, 91, 92, 93, 94, 95, 96, or 97% purity.

Still referring to FIG. 5, the relatively pure form of THC presents challenges in quantitative handling as the relatively pure form of THC has a high viscosity, such as like honey, and is concentrated enough that 10 mg of a pure THC concentrate is widely considered as a dose of THC by a consumer. Indeed, some states, such as Nevada, currently regulate a serving of THC in solution as being 10 or 12 mg of THC. To ease handling, the THC is optionally diluted 520 with an alcohol 522, such as ethanol; an 524 oil, such as canola oil, olive oil, and/or a medium-chain length triglyceride (MCT) oil; and/or with water 526. Generally, any solvent is used for dilution. The dilution aids injecting or spraying THC onto the original product 112 as adding pure THC necessitates additions of less than 25, 20, 15, 10, 5, or 2 μL of the relatively pure form of THC, where additions of such volumes lead to increasing percent errors as the volume of addition of the THC decreases, such as from 1 to 1.5, 2, 5, 10, or 20 percent error at the cited volumes. However, additions of larger volumes of a diluted THC stock 500 lead to reduced errors, which are typically less than 0.5, 1, or 2 percent. In addition, moving the honey like relatively pure THC with a honey like viscosity of 1,000 to 20,000 centipoise results in still higher quantitative errors due to the relatively pure THC sticking to the equipment used in quantitative volume transfer of a liquid, such as a pipette and/or the end of a delivery tube, such as an injector, dropper, or sprayer. However, the dilution of the relatively pure form of THC, such as with the alcohol 522 and/or the oil 524, reduces the error back down to the sub-one percent level as the viscosity may be adjusted down to less than 500, 100, 50, 25, 15, or 12 mPa sec, which is readily transferred with chemistry laboratory techniques, such as use of a micropipette and/or is readily delivered with a dropper, sprayer, and/or injector.

Herein, by definition, a compound combines separate things to form something. For example, still referring to FIG. 5, tetrahydrocannabinol and ethanol are combined to form a compound, such as a THC-alcohol compound. The compound is optionally and preferably further treated, such as through the application of heat to bring the compound to a temperature exceeding 20, 25, or 30 degrees Celsius or greater than 60, 80, 100, 120, 140, 150, 160, of 170 degrees Fahrenheit and preferably less than 175, 180, 200, 220, or 250 degrees Fahrenheit. Before, during, and/or after heating and optionally without heating, the THC-alcohol compound is optionally: mixed with a mixer, subjected to ultrasonic heating, and/or is subjected to shear forces, such as by passing through a high shear mixer, such as by techniques optionally used to process an emulsion, further described infra. Optionally and preferably, the THC-alcohol compound comprises less then 90, 80, 70, 60, 50, 40, 30, 20, 10, 5, 4, 3, 2, or 1 percent water by mass. Optionally, the only water in the THC-alcohol compound comes from water in an ethanol stock solution. A ratio of the tetrahydrocannabinol to ethanol is optionally greater than 1:100, 1:10, 1:2, 1:1, 2:1, 10:1, or 100:1 by volume and/or by mass. A ratio of the ethanol to tetrahydrocannabinol is optionally greater than 1:100, 1:10, 1:2, 1:1, 2:1, 10:1, or 100:1 by volume and/or by mass. By treating the THC-alcohol compound with the high shear mixer, as described infra, the THC-alcohol compound may form micelles of THC in the alcohol, lipid bilayers of THC in the alcohol, or other emulsion like forms, where the THC and alcohol maintain some distinction as separate species and/or where the ethanol takes the place of water, at least in part, in a THC/ethanol emulsion, such as where THC acts partially like an oil and ethanol has some polar forces like water. Application of shear forces to the THC-alcohol compound and/or the THC/ethanol emulsion results in smaller particle sizes with: increasing shear force, an increased time period of applied shear force, and/or with multiple passes through a high shear mixer, as further described infra. With sufficient time and/or passes in/through a high shear mixer, such as with shear forces exceeding 50,000, 100,000, 500,000, 1,000,000, 5,000,000, or 10,000,000 sec⁻¹, the resulting formulation becomes white, translucent, and/or clear and eventually yields a blue or purple coloration, which is indicative of mean particle sizes less than 2000, 1500, 1000, 800, 600, 500, 400, or 300 nm in mean diameter even after excluding particle sizes less than 10, 20, 40, 50, 75, or 100 nm in mean diameter. The unit sec⁻¹ is a simplification or reduction of a shear force measured in velocity/distance, such as resulting from a velocity of the compound, such as in meters/sec through a cross-sectional distance through which the compound/emulsion flows, such as in meters. For instance vel/distance is meters/sec/meters or sec^{-1.}

Viscosity

Still referring to FIG. 5, the stock additive 500 or a resulting suspension, emulsion, mixture, and/or compound is optionally and preferably added to and/or combined with a beverage 724, an edible 725, and/or a product 726, such as a preroll, also referred to as a pre-roll, or a filling solution for a vape pen. In a first process 541, the viscosity of the THC is reduced, such as from greater than 250, 500, 1000, 2500, 5000, or 10,000 cP to less than 1000, 500, 250, or 200 cP, such as be heating, adding an alcohol to the THC, such as ethanol, and/or adding a fat to the THC, such as a butter and/or a triglyceride. In a second process, the viscosity is further reduced 542, is increased 543, and/or is maintained 544. For instance, a viscosity of a resulting THC compound, such as a THC-ethanol compound, a THC-fat compound, and/or a THC-butter compound, with or without an emulsifying agent, is further reduced 542 by combining the THC compound with at least one beverage component, such as to a viscosity of less than 200, 100, 50, 25, 20, 10, 5, or 2 cP. Similarly, in a second process, the viscosity of the resulting THC compound is optionally increased 543, such as by combining the THC compound is an edible or by driving off the solvent, such as with a partial vacuum, to yield an edible with a viscosity greater than 200, 500, 1000, 5000, 10,000, or 20,000 cP. Similarly, in a second process, the viscosity is further altered 544, such as by combining the THC compound with a product, such as a vape pen solution or a preroll, which respectively decreases or increases the viscosity.

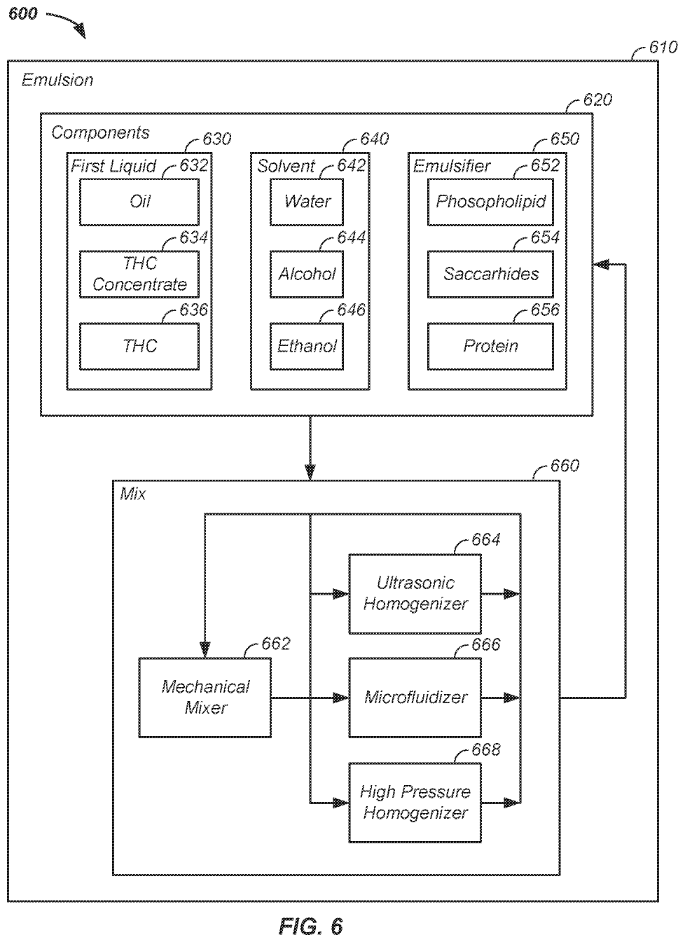

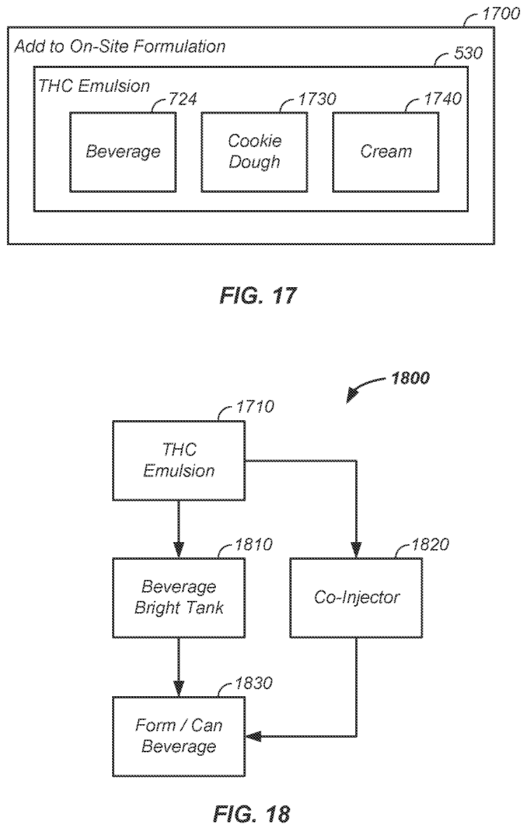

Still referring to FIG. 5 and referring now to FIGS. 6 and 17, optionally and preferably a THC emulsion 530 containing the THC 210 is formed. Formation of an emulsion 600 is further described infra. The THC emulsion 530 is optionally formed through addition of an emulsifier and an aqueous solution to one or more of the THC concentrate 510 and the diluted THC stock 520. Optionally, a step of adding nootropics 540 to any of the THC concentrate 510, diluted THC 520, and/or a THC emulsion 530 is performed in the formulation of the THC stock 500. As described, infra, the THC emulsion is subsequently injected into, dropped onto, mixed with, and/or sprayed onto the original product 112 in the formation of the amended product 114. The inventors have discovered that selection of chemical properties of the emulsifying agent to match a surface and/or a volume of the original product 112 facilitates adsorption of the THC onto the original product 112 and/or absorption of the THC into the original product 112 in the formation of the amended product 114. For instance, an emulsion of the THC 210 and a saccharide emulsifier aids adsorption of the THC 210 onto a starchy product, such as a chocolate or cheese puff and an emulsion of the THC 210 and a protein emulsifier aids adsorption of the THC 210 onto an oily product, such as some forms of oily potato chips. Notably, the inventors have discovered that a formulation of an emulsifier and THC 210 without an aqueous solvent and/or less than 30, 20, 10, 5, 2, or 1 percent water facilitates stickiness of the THC 210 onto a number of starchy, oily, and sweet, and savory products as the accessible portions of the emulsifier are attracted to the product and/or the water is internalized in cells within the oil.

Emulsion

The stock additive, described supra, which optionally contains THC and/or a nootropic, is optionally in the form of an emulsion. The emulsion is optionally injected into, dropped onto, and/or is sprayed onto the original product 112 to form the amended product 114 and/or is integrated into a formulation, such as in the formation of a beverage or a baked good.

Liquid Emulsion

Referring now to FIG. 6, an emulsion formation process 600 of forming an emulsion 610 is further described. A liquid emulsion is a system comprising two immiscible liquids where one liquid is dispersed in another, such as through use of an emulsifying agent. In examples herein, a first liquid 630, such as an oil, is suspended in a solvent 640, such as water. However, the water is optionally suspended in the oil. For clarity of presentation and without loss of generality oil in water emulsions are described.

Still referring to FIG. 6, generally components 620 of the emulsion 610 are combined and mixed 660. In one case, all of the components 620 of the emulsion 610 are combined and then mixed. In another case, at least some of the components 620 of the emulsion 610 are combined and mixed; at least some additional components are added to the mixture and the resulting mixture is further mixed, where any of the emulsion components 620 are added in the second step and the second step is repeated until all of the components 620 are added.

Still referring to FIG. 6, particular components 620 of the emulsion 610 comprise: (1) the first liquid 630, such as an oil 632, a THC concentrate 634, THC 636, and/or a nootropic; (2) the solvent 640, such as water 642, an aqueous solution, an alcohol 644, and/or ethanol 646; and (3) an emulsifier 650, such as a phospholipid 652, lecithin, a saccharide 654, a polysaccharide, gum Arabic, inulin, a modified starch, tween, cellulose, pectin, a protein 656, a gelatin, a caseinate, SPI, a dairy product, soy protein, whey protein, pea protein, a plant protein, and/or a chitin nanoparticle. Herein, for clarity of presentation and without loss of generality, emulsions of THC oil and/or an oil containing THC in an aqueous solution is described where the emulsion is formed with an emulsifier 650, such as lecithin.

Still referring to FIG. 6, an emulsifier is any one or more substances that stabilize an emulsion. Generally, an emulsifier has a first portion, such as a first portion of a molecule, that prefers to be in a first liquid, such as an oil, and a second portion, such as a second portion of the molecule, that prefers to be in a second liquid. An example of an emulsifier is lecithin. Lecithin is amphiphilic as it has a fat attracting portion or a lipophilic portion and a water attracting portion or a hydrophilic portion. Commercially available lecithin is a mixture pf phospholipids. Lecithin is an example of a glycerophospholipid. A glycerophospholipid is any derivative of glycerophosphoric acid that contains at least one O-acyl, or O-alkyl, or O-alk-1′-enyl residue attached to a glycerol moiety. Typically, lecithin has a glycerin backbone. In practice, lecithins are mixtures of glycerophospholipids including one or more of: phosphatidylcholine, phosphatidylethanolamine, phosphatidylinositol, phosphatidylserine, and phosphatidic acid. Lecithin is amphiphilic as lecithin attracts both water and fat substances.

Still referring to FIG. 6, at some point sub-components and/or all of the components 620 are mixed 660. Energy of a mixer translates to particle size of an emulsion. Typically, a step of mixing 660 the components 620 is done with one or more of a mechanical mixer 662, such as a home kitchen mixer, a home kitchen emulsifier, and/or a rotator-stator mixer. Sole use of a home kitchen mixer, which mixes at a rate of 0 to 2500 revolutions per minute (rpm), results in an emulsion having large particle sizes that separate in minutes to a few hours, which is however sufficient for some drop, spray, and/or injection processes for THC addition to the original product 112 to form the amended product 114, as further described infra. A home use kitchen emulsifier steps up rotation speed of the mechanical mixer to 6,000 to 20,000 rpm. Still faster mixing rates tend to degrade the DNA. A key feature of a home use kitchen emulsifier is that the mixing blade has a separation distance to a safety shroud of 0.5 to 5 or more millimeters. The home use kitchen emulsifier forms a more stable emulsion. However, the resulting micelles are still 2,000 nm or larger, which results in visible separation of an oil phase, like THC, and an aqueous phase within 24 hours. Again, this level of mixing is sufficient for some amendment tasks, but is insufficient for preparation of a stable THC in a beverage, such as in a soda and/or a carbonated beverage. A higher quality rotator-stator mixer is an advanced mechanical mixer that mixes at the same rates as a high quality home emulsifier. However, the rotator-stator mixer spins a rotator within a static housing, where the separation of the rotator and the stator is less than 2, 1, 0.5, or 0.1 mm. Resulting shear forces on the components 620 between the rotator and the stator form micelles with mean diameters of 1,000 to 2,500, which results in a milky emulsion that is stable for greater than 6, 12, 18, or 24 hours, but typically show beginning separation within 1, 2, 3, or more days. An ultrasonic mixer 664 creates cavitation bubbles that break apart and form still smaller micelles of the emulsion components, such as THC and emulsifier, of greater than 300 nm, but smaller in diameter than result from the rotator-stator mixer. Two additional systems, a microfluidizer 666 and a high pressure homogenizer 668, each create still smaller micelles of THC and emulsifier in water, such as less than 300, 200, 100, or 90 nm mean diameter, excluding micelles of less than 20 nm diameter. Optionally and preferably, the homogenizer, such as the high pressure homogenizer applies a shear stress greater than 100,000 sec⁻¹, 500,000 1/sec, or 1,000,000 1/sec to the emulsion, where a shear stress unit of force/area reduces to sec⁻¹. The smaller diameter micelles are stable for months and are optically clear, which are both distinct advantages in clear sodas or drinks. Herein, a measure of clarity is optionally obtained with a red laser, where an optically clear beverage widens a red laser beam by less than three hundred percent at a full width half height over a pathlength of ten millimeters. Nanoparticles, despite colloquial usage of the term, contain micelles with a mean diameter of less than 1000 nm, excluding micelles of less than 20 nm in diameter. Hence, the ultrasonic homogenizer 664, the microfluidizer 666, and the high pressure homogenizer 668 form nanoparticles-sized emulsions, while the home mixer, home emulsifier, and rotator-stator mixer do not. Further, the microfluidizer 666 and the high pressure homogenizer 668 form emulsions that are clear and/or are stable for months or years. In preparation of THC containing emulsions, the inventors have identified several combinations of mixing steps that form clear and stable emulsions, such as with a sequence of mixing that starts with one or more of the mechanical mixer 662, optionally uses the ultrasonic homogenizer 664, and concludes with one or more of the microfluidizer 66 and the high pressure homogenizer 668. Further, the process of adding components 620 and mixing 660 is optionally iterative for n=2, 3, 4, 5 or more iterations. The inventors have determined that operating a rotor-stator mixer at greater than 5000 rpm and less than 13,000 rpm reduces bubble formation and results in enhance stability of emulsions that are later processed with an ultrasonic mixer.

THC/Emulsifier Combination

Notably, an emulsifier free emulsion is optionally used to deliver THC to the original product 112. For example, the THC is suspended, at least temporarily, in a second liquid, such as the solvent 640, with or without the use of any emulsifying agent. Further, an emulsion is optionally formed with: (1) THC and/or a THC/oil mix and (2) an emulsifier without the solvent 640 or with minimal solvent, such as less than 10, 5, 2, 1, 0.5, or 09.1 percent water.

Still referring to FIG. 6, the inventors have discovered that THC/emulsifying agent emulsion without the aqueous phase allows quantitative dilution of the THC, as described supra, and a low viscosity solution that aids transfer of the THC, as described supra. Further, in the low/no aqueous phase emulsion formulation, dubbed a solvent free emulsion, properties of a selected emulsifying agent facilitate adsorption onto and/or absorption into the original product 112. Two examples further illustrate the concept of examining the chemical structure of the original product 112 and selecting a compatible chemical structure of the emulsifier in a THC/selected emulsifier solvent free emulsion to aid adsorption and absorption of the THC into the original product 112 to form the amended product 114.

### Example I

In a first example, the original product 112 has a starchy outer layer or a permeable starchy structure, such as a cheese puff. The starchy/non-oily structure of a cheese puff chemically and physically attracts (like attracts like chemical principle) polysaccharide emulsifier of a THC/polysaccharide emulsifier solvent free emulsion. More particularly, the polysaccharide emulsifier, encasing the THC oil, attracts to the starchy/dry surfaces of the cheese puff, such as both on an outer perimeter of the cheese puff and within a porous/permeable structure of the cheese puff. Said again, the accessible polysaccharide surface of a THC oil/polysaccharide low solvent or no solvent emulsion adsorbs onto and/or absorbs into the cheese puff as the chemical properties of the polysaccharide emulsifier chemically attract to the accessible surfaces of the cheese puff, which forms a stable and/or homogenous THC coating.

### Example II

In a second example, the original product 114 has an oily outer layer, such as an oily chip or potato chip. The oily structure/surface of the oily chip chemically and physically attracts (like attracts like chemical principle) an oily emulsifier of: (1) a THC/protein emulsifier solvent free emulsion or (2) simply THC cut with an oil. More particularly, the oil attracting surface of a protein emulsifier, encasing the THC oil, the THC oil, and/or an oil used to cut/dilute THC attracts to the oily surfaces of the chip, such as both on an outer perimeter of the chip and within a porous/permeable structure of the chip. Said again, the accessible oily surface of pure THC, a THC cut with oil, and/or THC/protein emulsifier with little to no solvent adsorbs onto and/or absorbs into the oily chip as the oily surfaces attract oily surfaces, which forms a stable and/or homogenous THC coating.

Add Additive to Product

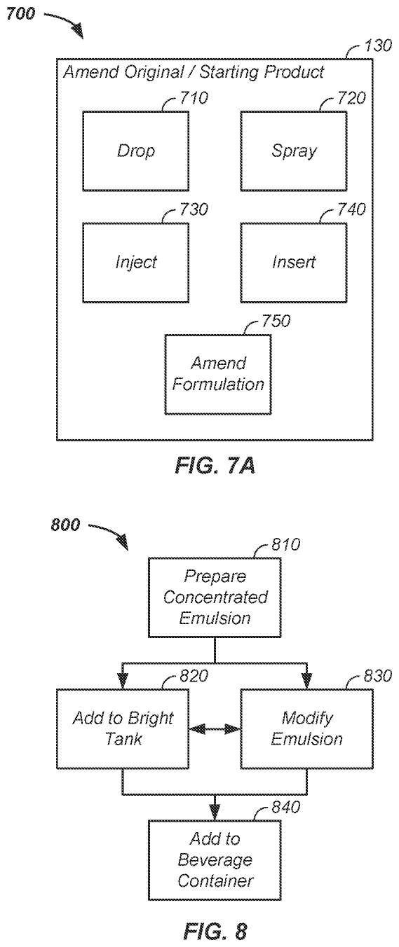

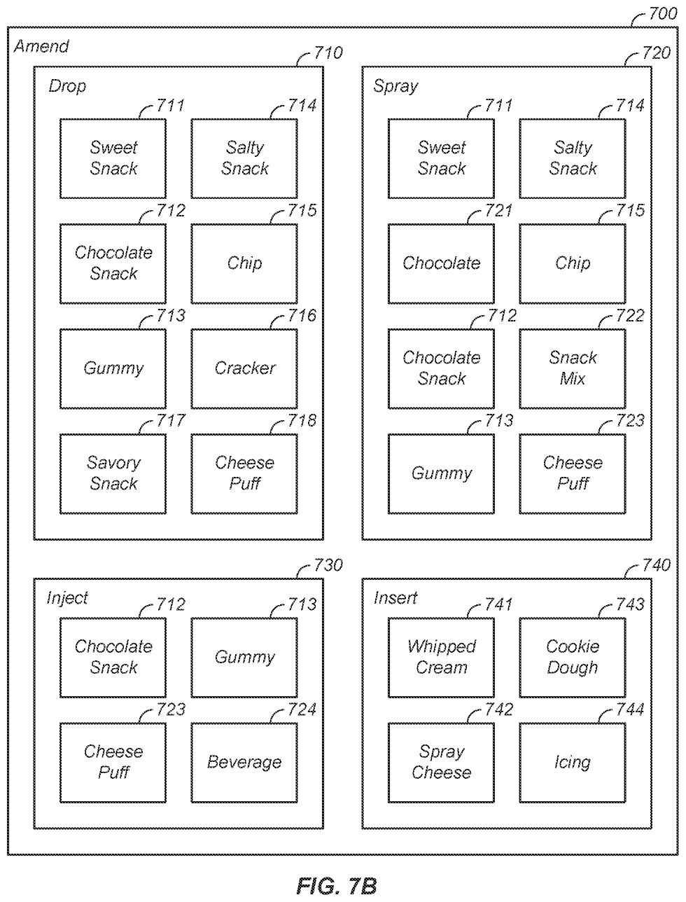

Referring now to FIG. 7A, FIG. 7B, and FIG. 8, processes of amending the composition/original product 130 are described. For example, the original product 112 is amended to include one or more of THC and/or a nootropic.

Referring now to FIG. 7A, five optional processes of amending the composition/original product 130 are described. Again for clarity of presentation and without loss of generality, the amendment processes use examples of amending with THC. A first amendment process adds THC to the original product 112 by dropping 710 THC and/or a mix containing THC onto the original product 112 to form the amended product 114 now containing THC. Generally, the THC, in the form of a liquid or a solid, is dropped onto the original product 112. For instance, crystallized THC is dropped onto the original product 112. In another case, a liquid and/or an emulsion containing THC is dropped onto the original product 112. For example, a liquid containing THC is mechanically pumped onto individual elements of the original product 112, without a human physically/directly moving a pump dispersing element of the semi-automated or automated pump. A second amendment process adds THC to the original product 112 by spraying 720 THC and/or a liquid solution containing THC onto the original product 112 to form the amended product 114 now containing THC. For instance a chip, pretzel, and/or gummy is sprayed with the THC containing solution and/or the THC containing emulsion. A third amendment process adds THC to the original product 112 by injecting 730 THC and/or a liquid solution containing THC into the original product 112 to form the amended product 114 now containing THC. For instance, cream filled chocolate, a liquid filled chocolate, and/or a gummy is injected with the THC, THC containing solution, and/or the THC containing emulsion. A fourth amendment process adds THC to the original product 112 by inserting 740 THC and/or a liquid solution containing THC into the original product 112 to form the amended product 114 now containing THC. For instance THC is inserted into a canister of liquid cheese, a whipped cream container, a tube of cookie dough, and/or icing. In all cases, the THC is adsorbed onto the surface of the original product 112 and/or the THC is absorbed into the original product 112 to form the amended product 114, now containing THC. In all cases, the THC is optionally and preferably mechanically added to the original product 112 without direct handling of the application apparatus by a human. For instance, that applicator is under computer control and uses a pump, volume delivery system, atomizer, sprayer, mister, injector, syringe, and/or dispenser. A fifth amendment process amends a formulation 750. For instance, THC, a THC stock, and/or a THC containing emulsion is added to a formulation before packaging as the amended product 114. For instance, the THC is added to an original product of a syrup mix used to make sodas and/or is added to a brite tank of a drink ready for canning or bottling. A soda mix includes at least water, a sweetener, a flavoring, and a preservative. A soda mix is optionally and typically available for sale in its own packaging. Each of the amendment processes is further described infra.

Referring now to FIG. 8, a preparation/packaging system 800 is illustrated, for preparing a product for canning. As illustrated, in one step a concentrated emulsion 810 is formed, such as containing at least THC 210. Optionally and preferably, the concentrated emulsion contains at least 1000, 2000, 10,000, 25,000, or 50,000 mg THC per twelve ounces. For example, the concentrated emulsion is prepared using any 1, 2, 3, or more of the mixing techniques described infra. In a later step, the prepared concentrated emulsion is added to a bright tank 820. Herein, a bright tank is sometimes referred to as a “brite” tank, which is a tank used to hold clarified/non-cloudy beer. However, generally, the bright tank refers to a holding vessel and/or a dilution vessel, which is optionally pressurized, such as with air, carbon dioxide, nitrogen, and/or nitrous oxide. Upon adding the concentrated emulsion to the bright tank, the concentrated emulsion is diluted by a solution already in the bright tank, such as an aqueous solution, and/or the concentrated emulsion is modified 830 after addition to the bright tank, such as by dilution with an aqueous solution. Optionally and preferably, a resulting modified emulsion has a THC concentration of less than or equal to 500, 250, 200, 100, 50, 20, or 10 mg THC/twelve ounces. The inventor notes that preparing a diluted concentration of THC in an emulsion, such as less than 1000 mg/twelve ounce can is vastly inefficient as the mixing processes operate on a sub-volume of the total volume. Particularly, the emulsion is formed at the mixer interface, such as a blade or tube and as the solution is diluted, the formation of the emulsion becomes less efficient as the constituents to be emulsified contact the blade or tube less often. Thus, a preparation, such as with a high-shear mixer, of a concentrated emulsion of 50,000 mg THC/twelve ounce that takes one hour would take 1000 hours if at 50 mg THC/twelve ounce can. Similarly, the 50,000 mg THC/twelve ounce can makes 1000 cans of 50 mg THC/twelve ounce can, where 1000 hours is 42 days. Certainly, in a normal production of 1,000,000 cans this becomes impossible mixing diluted product, where 1,000,000 cans in this example leads to a mixing time of 114 years without the reduction in concentration step. Thus, the preparation of a concentrated emulsion has an industrial benefit in terms of energy use, time, scaling, and floor size requirements. The resulting emulsion optionally contains any of the additive described herein, such as any alcoholic beverage, soda mix, nootropic, ethanol, flavoring, and/or preservative. The resulting emulsion optionally comprises any chemical and/or physical characteristics described herein, such as clarity, viscosity, and/or concentration. In a subsequent step, the resulting emulsion is added to a beverage container 840 and sealed, such as with a production canning line and/or with a pump. Herein, a bright tank is at least 40, 90, 140, 200, 500, or 900 gallons. Optionally and preferably, the dilution of emulsion is at least 10, 50, 100, or 1000 parts diluent to 1 part emulsion (10, 50, 100, or 1000:1). Optionally and preferably a soda pre-mix, such as a prepacked mix of at least water, a sweetener, a flavoring, and a preservative is used as a portion of the diluent, where the pre-mix-to-concentrated emulsion ratio is at least 1, 2, 3, 4, 5, or more part pre-mix-to-one part concentrated emulsion, where the emulsion is used to deliver the THC 210 into the resulting solution as a known concentration, homogeneously, and/or in a stable/non-separating resulting diluted emulsion. Optionally and preferably, the bright tank is pressurized with at least one of carbon dioxide, nitrogen, and nitrous oxide at a concentration at least double that of the atmosphere, to a pressure in excess of at 3, 5, or 10 pounds per square inch for a period in excess of 0.5, 1, 2, 3, or 5 hours.

Referring now to FIG. 7B, additional examples of amending, spraying, injecting, and inserting THC 210 and/or an amendment additive 200 are provided. As illustrated, the technique of dropping 710 THC 210 onto an original product 112 to form the amended product 114 is optionally used to add THC 210 and/or the amendment additive 200 to any of: a sweet snack 711, such as a chocolate snack 712 or a gummy 713; a salty snack 714, such as a chip 715, cracker 716, or Chex mix; and/or to a savory snack 717, such as a cheese puff 718, cheese flavored chip, cheese flavored cracker, or cheese. Similarly, as illustrated, the technique of spraying 720 THC 210 onto an original product 112 to form the amended product 114 is optionally used to add THC 210 and/or the amendment additive 200 to any of: a sweet snack 711, such as chocolate 721 or a chocolate bar, a chocolate snack, a candy, a caramel, and/or a gummy; a salty snack 714, such as a chip 715, a snack mix 722, and/or a cheese puff 723. Similarly, as illustrated, the technique of injecting 730 THC 210 onto and/or into an original product 112 to form the amended product 114 is optionally used to add THC 210 and/or the amendment additive 200: into a chocolate snack 712, into a gummy 713, into a cheese puff 723, and/or into a beverage 724. Similarly, as illustrated and further described infra, the technique of inserting 740 THC 210 onto and/or into the original product 112 to form the amended product 114 is optionally used to add THC 210 and/or the amendment additive 200 to any of: whipped cream 741, spray cheese 742, cookie dough 743, and/or icing 744. Notably, one or more amendment approaches work for a common food/beverage item, such as one may drop onto, inject into, and/or spray a gummy. Generally, any of the amendment approaches of dropping 710, spraying 720, injecting 730, inserting 740, and/or amending a formulation 750 are optionally used on any food product and/or with any beverage product, such as soda, an alcoholic drink, and/or coffee, albeit with differing outcomes of stability.

Still referring to FIGS. 7 and 8 and referring now to FIG. 18, a selected addition technique and/or a selected chemical/physical make-up of the THC/additive solid, crystal, solution, mixture, homogeneity, and/or emulsion is optionally and preferably dependent upon the chemical/physical properties of the make-up of the food/drink product to be amended. For instance, an aqueous drink product will naturally separate from oily THC, so the THC is optionally and preferably emulsified so that when the THC emulsion is added to the aqueous solution, the THC containing droplet, cells, and/or micelles disperse to form a homogenous solution as opposed to clumping together and floating to the top of the aqueous solution, such as in a soda. Indeed, many states require homogenization of the THC in the food/beverage product. For instance, Nevada requires that each serving of a THC containing fluid, such as a soda, have no more than 10 mg THC. Thus, if 40 or 50 mg of THC are added to a soda and/or are formulated into a soda that is labeled as having 4 or 5 servings per container, such as an 8, 12, 16, or 20 ounce soda container, then the THC must be homogenous in the soda as if the THC separates or clumps and floats in the soda, one serving may have greater than 90 or 95% of the 40 or 50 mg of THC, which is illegal and may not be safe for some consumers. Similarly, if a food product is selected that has an oily coating, then dropping a liquid emulsion of THC onto the oily food product is likely to result in the THC emulsion running off of the oily food product, which results in an unsatisfied consumer who did not receive the THC on the food product as it is left as a coating inside a shipping container, such as a bag. Thus, a better addition selection for the food product with an oily coating is: (1) injection of: THC 210, the THC stock 500, the diluted THC 520, and/or a THC emulsion 530 into the food product with the oily surface if the food product with the oily surface contains a cavity, a liquid center, is porous (like a cheese puff), and/or is readily amendable to rapid internal diffusion, such an air whipped chocolate filling; (2) spraying of: THC 210, the THC stock 500, the diluted THC 520, and/or the THC emulsion 530 onto the food product with the oily surface if the food product with the oily surface is adsorbent enough to adhere to the THC oil and/or if the food product with the oily surface is porous (like a cheese puff or soft cracker). Generally, the chemical outer surface of the selected form of THC (concentrate, oil dilution, or emulsion) is optionally and preferably matched chemically to an accessible surface of the food product (THC oil to accessible food oil or THC water emulsion to accessible food water). Similarly, the selected form of the THC (concentrate, oil dilution, or emulsion) is optionally and preferably matched with porosity of the selected food product. For instance, an oil thinned THC solution or even a thinned THC emulsion will penetrate well into a porous food product while the THC concentrate 510 may be too viscous to penetrate into the porous food product. Optionally and preferably, the THC stock 500, the diluted THC 520, and/or a THC emulsion 530 is created to have a viscosity of greater than 1 and less than 10, 12, 15, 20, 30, 50, 100, 1000, 5000, or 10,000 mPa sec or centipoise or an equivalent measure in centistokes.

Automated Production

In the THC production world, THC containing products are made one at a time by hand. For instance, a worker takes a syringe of THC and injects raw THC into a gummy. At best, a worker bakes a tray of brownies infused with raw THC. As a result, costs are high, precision is poor, accuracy is bad, and homogeneity of THC within a product is terrible. This is largely due to no consideration of chemical THC properties in relation to a food product, which typically results in large, even illegal, amounts of THC in one serving and essentially no THC in another serving within a single package. Further, no automation exists, such as used in international and/or national production facilities, as it is illegal to ship across state lines any THC product. Thus, the massive production facilities for commonly available products may not be used to produce THC containing products as distribution of the THC containing product across state lines is currently, as of 2020, illegal in the United States.

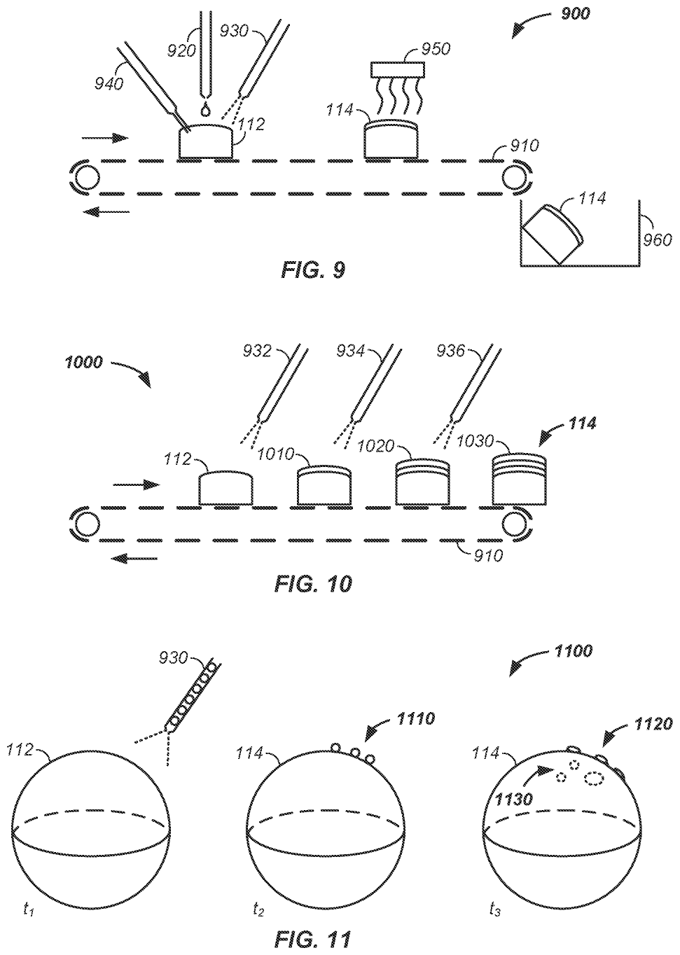

Referring now to FIG. 9, a semi-automated/automated amendment production line system 900 is described. Again, for clarity of presentation and without loss of generality, examples herein use THC as a representative additive. Generally, mass production techniques are modified for THC addition to the original product 112 to form the amended product 114, as further described infra.

Still referring to FIG. 9, the semi-automated/automated amendment production line system 900 optionally and preferably moves the original product 112, such as on a conveyor belt 910 past: (1) a dropper 920, which is part of a dropper system used to perform the task of dropping 710 the THC onto the original product 112 to form the amended product 114; (2) a sprayer 930, which is part of a sprayer system used to perform the task of spraying 720 the THC onto the original product 112 to form the amended product 114; and/or (3) an injector 930, which is part of an injector system used to perform the task of injecting 730 the THC into the original product 112 to form the amended product 114. Optionally, a dryer 950, such as a heating system is used to dry the THC additive once dropped onto and/or sprayed onto the now amended product 114. Optionally and preferably, the amended product 114 is automatically packaged with a packaging system 960, such as an automated bagging system, an automated boxing system, and automated canning system, and/or an automated bottling system. Optionally and preferably, no human is handling/holding the original product 112, any element of the dropping system, any element of the sprayer system, and/or any element of the injector system while the additive, such as THC, is applied to the original product 112 to form the amended product. Further, optionally and preferably, the packaging system 960 functions without any human touching the amended product 114 or the container into which the amended product 114 is packaged at the time of packaging. Optionally and preferably, a human operator operates a computer controlled controller at an operating station, where the computer then controls the amendment and/or the packaging steps. Examples are provided, infra, that further describe the semi-automated/automated amendment production line system 900.

### Example I

Referring now to FIG. 10, a first example of the semi-automated/automated amendment production line system 900 is provided. In this example, a system of multiple amendments 1000 to the original product 112 is illustrated. As illustrated, the original product 112 is sprayed multiple times with the spraying system. However, multiple additives are optionally dropped onto, sprayed onto, and/or injected into the original product 112, with the dropper 920, the sprayer 930, and/or the injector 940 respectively. For instance, a first sprayer 932 optionally sprays a bonding agent, such as a gum containing solution; a second sprayer 934, simultaneously or at a later time, sprays on the additive, such as the THC stock solution 500; and a third sprayer 936, simultaneously or at a later time, sprays on another binding agent layer or a sealing layer, such as a chocolate or edible waxy coating. As illustrated, sequential uses of the first sprayer 932, the second sprayer 934, and the third sprayer 936 respectively form a first coating layer 1010, a second coating layer 1020, and a third coating layer 1030 on the now amended product 114. In one case, the middle second coating layer 1020 contains THC, which is held to the original product 112 by a binding layer, such as the first layer 1010, and is optionally sealed onto the amended product 114 by the optional sealing layer, the third layer 1020, which is optionally another binding layer. As the first, second, and third layers are optionally co-sprayed and/or are miscible while still wet, the three layers optionally and preferably mix and bind. Generally, the sprayer adds any number, n, layers, such as 1, 2, 3, 4, 5, or more layers. The sprayer 930 is optionally attached to a high pressure emulsifier, such as directly attached to an outlet of the high pressure emulsifier and/or attached with tubing to the high pressure emulsifier.

### Example II

Referring now to FIG. 11, a second example of the semi-automated/automated amendment production line system 900 is provided. As illustrated, the sprayer 930, which is optionally the dropper 920, quantitatively sprays, such as by volume, an amendment, such as a portion of the THC stock solution 500, onto the original product 112, which initially, such as at a first time, t₁, adsorbs 1110 and/or undergoes adsorption onto the surface of the now amended product 114. At a second time, the amendment, such as the applied portion of the THC stock solution 500, spreads 1120 on the outer surfaces of the amended product 114 and/or absorbs into and/or undergoes absorption into the amended product 114. The inventors have determined that matching chemical properties of the THC stock solution 500 to the accessible surfaces/volumes of the original product 112 facilitates the THC bonding to, adhering to, joining with, absorbing into the now amended product 114; aids retention of the THC on the amended product 114; and/or facilitates homogenous distribution of the THC on/within the amended product 114.

Injection

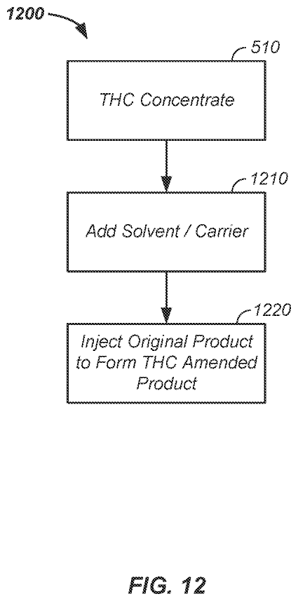

Referring now to FIG. 12, the process of injecting 730 is further described. An injection amendment process 1200 is illustrated in FIG. 12. Generally, the THC concentrate 510 is diluted by adding a solvent and/or a carrier 1210 to form a diluted THC solution and/or a THC emulsion, as described supra, which is easier to quantitatively handle, as described supra. The, now diluted THC, is subsequently injected 1220 into the original product 112 to form the amended product 114. In a case of injecting a cream filled chocolate, an internal liquid/paste of the cream filled chocolate preferably has a viscosity of less than 1000, 500, 200, or 100 mPa sec, which allows the diluted THC to adhere to and/or mix with the cream center and allows injection of the THC, such as a portion of the THC stock 500, into the chocolate without overflowing back out of the chocolate as is the case when trying to inject the THC into a highly viscous hard chocolate coating or into a highly viscous caramel, such as with a viscosity exceeding 1000 centipoise (cps), which is a measured viscosity of caramel candy. Optionally, a portion of the injector 940 contacting the original product, such as a chocolate shell, is elevated in temperature to a range of 80 to less than 140, 150, 160, 170, 180, 190, 200, 250, or 300 degrees Fahrenheit, which melts/lowers hardness/viscosity of the chocolate. More preferably, the injector is maintained at less than 200, 180, 170, 150, 130, 110, 102, or 100 degrees Fahrenheit as THC starts to evaporate at 150 degrees Fahrenheit and terpenoids start to evaporate at 102 degrees Fahrenheit.

### Example III

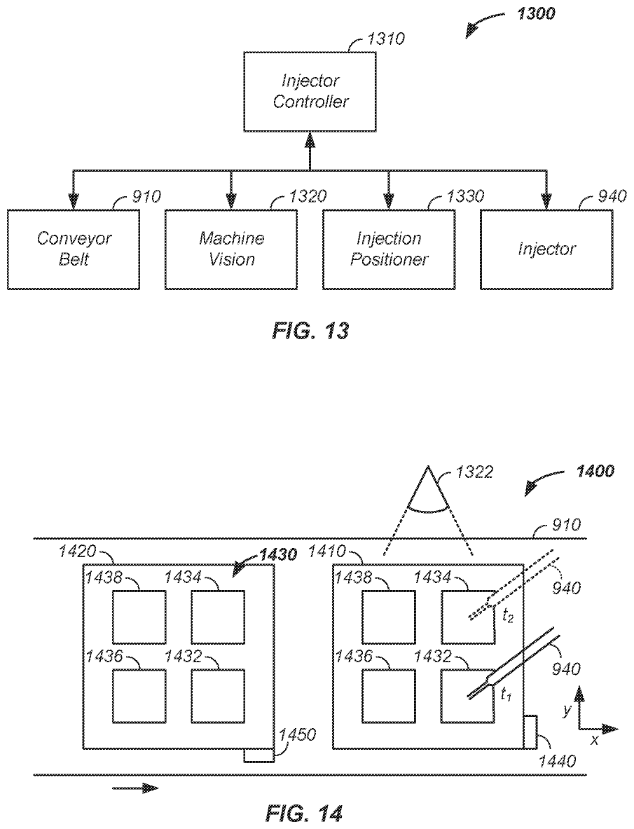

Referring now to FIG. 13, an injection system 1300, such as of the semi-automated/automated amendment production line system 900 is described. Optionally and preferably, the injection system 1330 includes an injection controller 1310, which controls and/or receives input from one or more of: the conveyor belt 910, a machine vision system 1320, an injection positioner 1330, and the injector 940. For instance, the injection system 1330 and/or a main controller that control the injection system and/or other sub-units of the semi-automated/automated amendment production line system 900, controls movement of the conveyor belt 910 to move a series of original products 112 to the injector 940. Optionally and preferably, the injector controller 1310 controls an injection positioner 1330, such as an x-, y-, and/or z-axis controlled injector, and a pump (not illustrated for clarity of presentation) linked the THC stock and to the injector 940 to move the injector 940, sequentially, from a non-delivery position, into contact with the original product 112, into the original product 112, and after the pump delivers the THC stock 500 or the like into the now amend product 114, out of and away from the now amended product 114. Optionally and preferably, the injector controller 1310 is aided with knowledge of a current position of one or more elements of the original product 112 through the use of one or more mechanical product guides, one or more mechanical product stops, and/or machine vision 1320, as further described infra.

### Example IV

Referring now to FIG. 14, a system of injecting multiple elements per tray, box, and/or container 1400 is illustrated. As illustrated, the conveyor belt 910 moves a first container 1410 into position, such as along the x-axis, where the first container 1410 is optionally guided/positioned by one or more guide rails 1450 and/or is positioned by one or more mechanical stops 1440. The illustrated first container 1410 and second container 1420 are two of a potentially endless line of containers. Each container contains a set of n original products 1430, where n is a positive integer of 1, 2, 3, 4, 5, or more. As illustrated, each container contains four original products, a first original product 1432, a second original product 1434, a third original product 1436, and a fourth original product 1438, such as chocolates. Optionally, a camera 1322 of an imaging system or the machine vision system 1320 informs the injector controller 1310 as to the location of each set of n original products 1430 as the original products near the injector 940. Optionally and preferably, the injector controller moves the injector 940 along x-, y-, and/or z-axes to inject, sequentially, to each of the original products, such as the first original product 1432 being injected at a first time, t₁, and the second original product 1434 being injected at a second time, t₂, which continues from item to item and from tray to tray.

Pressurized Container Amendment

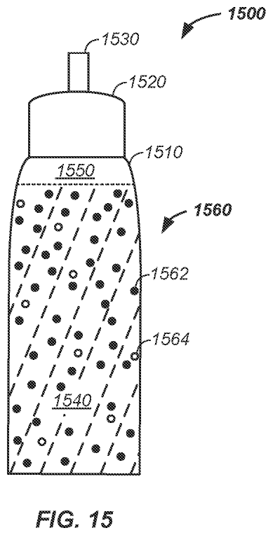

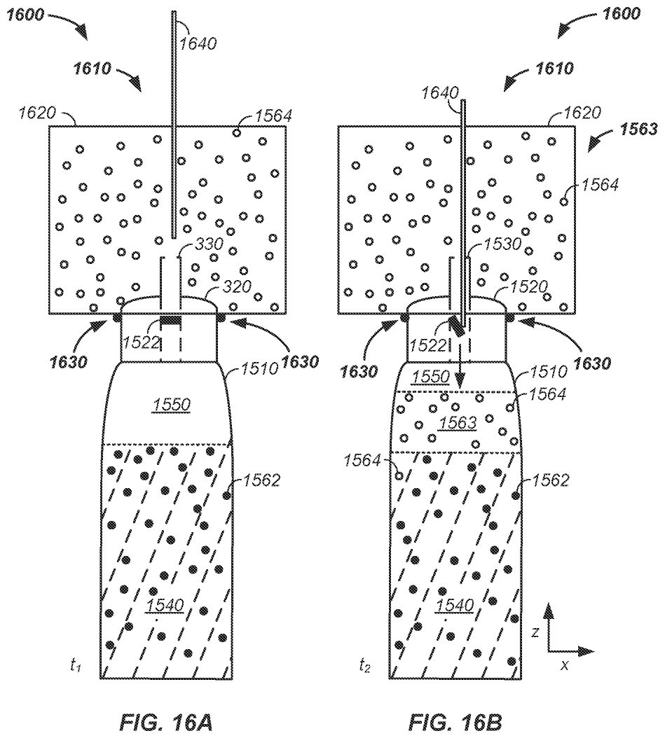

Referring now to FIG. 15, FIG. 16A, and FIG. 16B, an example of the insertion 740 method of the process of amending the starting product 130 is provided, where a pressurized product is amended.

### Example I

In a first example, referring now to FIG. 15, an amended pressurized product 1500 is illustrated, which is an example of an amended product 114. As illustrated, the amended pressurized product 1500 is packed in a pressurized container 1510, such as a canister. The pressurized container 1520 include a valve portion 1520, which in this case is illustrated in an upper portion of the pressurized container 1510. The valve portion 1520 contains a lever, valve, and/or port that is repetitively opened and closed by a user, such as to dispense the product 1500. As illustrated, the pressurized container 1510 contains a delivery port 1530.

For clarity of presentation and without loss of generality, in the second example, still referring to FIG. 15, whipped cream is used to describe the amended pressurized product 1500, which is dispensed from a pressurized container 1510. However, other products are optionally delivered from a pressurized container 1510, such as cookie dough, icing, a beverage, or spray cheese. While the pressurized container details will vary with product, such as going from an aerosol container to a canister with a piston wiper valve and a separated pressurized portion of the container, the concepts described herein of opening the pressure seal, amending the product, and resealing/re-pressurizing the container still apply.

In this first example, still referring to FIG. 15, the pressurized container is described in terms of zones and in terms of product constituents. First, the pressurized container 1510, as illustrated, contains a liquid/semi-solid zone 1540, such as liquid portion or high viscosity portion, and/or a gas zone 1550, such as a gas portion 1550. For instance, in the case of a whipped cream canister, the liquid portion is cream and the gas portion is a propellant, such as carbon dioxide, argon, a noble gas, butane, and/or preferably nitrous oxide. For clarity of presentation and without loss of generality, the gas is referred to herein as nitrous oxide. The propellant, such as nitrous oxide, resides in the gas portion 1550, which is also referred to as a headspace. In the case of nitrous oxide, which is similar to other gases, the nitrous oxide partially dissolves into the cream. When the cream, containing the nitrous oxide, moves from the pressurized contain 1510 to atmospheric pressure, the nitrous oxide expands. The expansion of the nitrous oxide expands/whips the cream into whipped cream. Second, the pressurize container 1520, as illustrated, contains the product 1560, which contains n constituents, where n is a positive integer of greater than 0, 1, 2, 3, 4, 5, or 10. As illustrated in this whipped cream example, a first constituent 1562 comprises cream and a second constituent 1564, such as THC. For clarity of presentation, the gases dissolved in the cream are not illustrated and components solvating, bonded to, adhered to, chemically bonded to, and/or mixed with the THC are not illustrated. As the cream is dispensed from the pressurized container, through the valve portion 1520, and optionally through the delivery port 1530, the THC is delivered in the resultant whipped cream.

In this first example, now referring to FIGS. 15, 16A, and 16B, the formation of the amended product in the container, such as the pressurized container 1510 is described. The pressurized container 1510 contains a valve portion 1520. Generally, the valve portion 1520 alternatingly allows passage of a contained component through the valve and stops passage of the contained component through the valve. Many types of valves exist, such as toggle, check, globe, plug, gate, globe, plug, ball, butterfly, check, diaphragm, pinch, pressure relief, Lindal, and/or control valves. Herein, all valve types are openable and closable. The valve is optionally positioned anywhere in the pressurized container 1510 and/or is affixed to the pressurized container 1510. As illustrated, the valve portion 1520 includes a flow control component 1522, which is a portion of any of the above listed valve types.

In this first example, referring still to FIGS. 16A and 16B, an amendment process 1600 includes attaching an amendment container 1610 to at least a portion of the pressurized container 1510 that is openable, such as the valve portion 1520 and/or the delivery port 1530. More particularly, a seal is formed between an output of the amendment container 1610 and an input/output of the pressurized container 1510. Typically, the valve portion 1520 of the pressurized container 1510 controls dispensing the product 1500 out from the pressurized container 1510, such as through the delivery port 1530. However, in the amendment process, flow through the valve portion 1520 is reversed. More particularly, amendment contents 1563 of the amendment container 1610, such as the second portion of the ingredients, described supra, sequentially pass from the amendment container 1610, through the valve portion 1520, and into the pressurized container 1510. In this manner, contents of the amendment container 1610 are transferred into the pressurized container 1510, which mix and/or react with the incomplete product and/or the product contained in the pressurized container 1510 to form an amended product, current product, updated product, modified product, the final product, a secondary product, and/or, after the addition of contents from the amendment container, simply the product 1500.

In this first example, still referring to FIGS. 16A and 16B, the amendment process 1600 temporarily opens a passage into the pressurized container, such as through the flow control component 1522. As described, supra, many valve types are optionally used. Further, many valve types include sub-options on how to open the valve. For instance, a toggle valve stem is pushed sideways to open up a toggle valve seal, where herein the toggle valve is an example of the valve portion 1520 and the toggle valve seal is an example of the flow control unit 1522. Further, the stem is a component of the valve. Similarly, a ball valve is another example of the valve portion 1520 operated by a lever and the ball with a hole in it that is turned in a ball valve is another example of the flow control unit 1522. More generally, any valve type is an example of the valve portion 1520 and any operable element of the valve type that controls flow, in and/or out, is an example of the flow control unit 1522. As illustrated, an opening/shutting control element 1640 operates on the valve portion 1520 to alternatingly open and close the valve, which controls flow of substance into and/or out of the pressurized canister 1510. Notably, the opening/shutting control element 1640 is in a first case built into the valve, such as a handle is built into a ball valve and a stem is built into a toggle valve. However, the opening/shutting control element 1640 is in a second case designed for use to open a valve flow control unit 1522 in a manner not originally designed into the valve type, as originally manufactured/sold. As illustrated, the opening/shutting control element 1640 is inserted into the valve portion 1520, optionally through the delivery port 1530, where the opening/shutting control element 1640 temporarily opens the flow control unit 1522. When the opening/shutting control element 1640 is withdrawn from contact with the flow control unit 1522, the flow control unit 1522 shuts and operation of the valve portion as manufactured is restored. The opening/shutting control element 1640 is optional when the built in mechanism of the valve portion 1520 includes a mechanical and/or an electromechanical element that is built in to control opening and shutting the flow control unit. In this case, the valve portion 1520 is optionally opened and/or closed using the originally manufactured control, such as a button, switch, stem control in the toggle valve example, and/or lever in the ball valve example. In this case, the opening/shutting control element 1640 is optionally used to operate the original control, such as through a robotic control. For instance, the opening/shutting control element is used to provide a sideways torque to the stem of the toggle valve or to rotate the handle in ball valve examples. Timing of operation of the opening/shutting control element 1640 is timed to injection/insertion of the amendment contents 1563 from the amendment container 1620 into the pressurized canister 1510, such as through a direct connection, an injection line or tubing. Generally, an attachment is made between the amendment container 1620 and the pressurized container 1510 through which the amendment contents 1563 flow and the opening/shutting control element 1640, timed with a desired flow of the amendment contents 1563 into the pressurized container 1510, opens and shuts the flow control unit 1522 of the valve portion 1520. For instance, a hose, through which the amendment contents 1563 flow, connects the amendment container 1620 to the pressurized container 1510 and in the case of a toggle valve, the opening/shutting control element provides a sideways pressure on the stem of the toggle valve to control when the amendment contents 1563 flow into the pressurized canister 1510.

In the first example, still referring to FIGS. 16A and 16B, as illustrated at the first time, the unamended product, such as the product shipped 428 from the first location 420 contains the liquid zone/high viscosity zone 1540 and the gas zone 1550. For the illustrative example of whipped cream, the liquid zone 1540 comprises cream and the gas zone 1550 comprises a propellant, such as nitrous oxide as described supra. Similarly, for a sprayable cheese product, the liquid zone/high viscosity zone 1540 comprises liquid cheese and/or semi-liquid cheese and there is essentially no gas zone in a food product chamber. At the first time, 6, the amendment container 1620 is attached to the pressurized container 1510. For instance a tube connects an output of the amendment container 1620 to an as yet still closed input element of the pressurized container. Optionally, the connection is air tight for the case of an already pressurized container. The connection could simply be gravity directing flow of output from the amendment container 1620 to the pressurized container 1510 in cases where the pressurized container is not yet pressurized and/or has not yet been sealed, such as in a process of fitting the valve portion 1520 onto and/or into the pressurized container 1510.

Optionally and preferably, at a second time, t₂, at least a portion of the amendment contents 1563 are transferred from the amendment container 1620 into the pressurized container 1510. As illustrated, during at least a portion of the second time, t₂, the opening/shutting control element 1640 functions to open the flow control unit 1522, as described supra. The delivery of the amendment contents 1563 to the pressurized container is driven by a force, such as: gravity, a pump, a timed pump, and/or a pressure differential. Optionally, delivery of the amendment contents 1563 additionally adds pressure and/or delivers a first pressure to the contents of the pressurized container 1510. For instance, the delivery of the amendment contents 1563 from a pressurized version of the amendment container 1620 is used to bring the pressure inside the pressurized container 1510 to a final shipping pressure of less than 200 psi, such as in a range of 140 to 180 psi.

Still referring to FIG. 16B, timing and flow of the amendment contents 363 is optionally and preferentially controlled and/or monitored with a flow valve. As illustrated, at the second time, t₂, the amendment contents 363 initially form, for a time period of less than 10 microseconds to a time period of greater than four hours, a zone that is not yet equilibrated or mixed into the liquid/semi-solid zone/high viscosity zone 340 and/or the gas zone 350. However, the amendment contents 363 mix with the liquid/high viscosity zone 340 and/or the gas zone 350 as further described, infra.

Herein, the second constituent 1564, such as THC, in the amendment contents 1563 is optionally and/or preferably in a natural form, in a purified form, in a liquid form, in a suspension, in a colloidal suspension, in a micelle, in a liposomal solution, dissolved in a solvent such as greater than 1, 2, 5, 10, 25, or 50 percent ethanol and/or greater than 1, 2, 5, 10, 25, or 50 percent butane, and/or is pre-homogenized to aid in mixing with the contents of the liquid/high viscosity zone 1540 of the pressurized container 1510. For instance, the inventors have discovered that THC dissolved in ethanol diffuses/permeates into liquid cheese to uniformly distribute the THC in the cheese product. Optionally and preferably, THC and/or THC in a solvent, such as ethanol, is a component of a formed suspension/emulsion, such as THC in water or THC in an aqueous mix, such as a beverage component. Optionally and preferably, the THC is emulsified in the water/aqueous mix along with one or more of: a surfactant, such as lecithin, an ester of glycerol, a Tween, such as Tween 20, 40, 60, or 80; a polysaccharide, such as gum Arabic, sap of an acacia tree, pectin, inulin, and/or Jujube polysaccharide; and/or a protein, such as soy protein, whey protein, pea protein isolate, and/or a gelatin.

Timing and/or volume of flow of the amendment contents 1563, which either produce directly or are used to calculate a volume of flow are additionally combined with a concentration to calculate/yield an amount of delivered product, such as milligrams of THC added to a container, such as the pressurized canister. The amount of THC is optionally digitally added to a certification report, which is optionally part of a certified and regulatory controlled chain of reports tracking THC along any portion from production, through isolation/extraction, to addition to a formulation, to distribution, and/or sale.

### Example II

In a second example, referring now to FIG. 16B and FIG. 15, the amendment contents 1563 are mixed into the liquid/semi-solid zone/high viscosity zone 1540 and/or the gas zone 1550 of the final product. As illustrated in FIG. 16B, after even a short time period, such as less than 1, 30, or 60 seconds, a portion of the amendment contents 1563, such as THC dissolve and/or move into the liquid/semi-solid zone/high viscosity zone 1540. Transfer of the amendment contents 1563, such as THC, from an amendment zone into the liquid/semi-solid zone/high viscosity zone 1540 of the final product is facilitated in a number of ways, such as any of shaking, heating, and/or stirring. For instance, after addition of the amendment contents 1563 into the pressurized canister 1510 and optionally and preferably after removing all connections between the amendment container 1620 and the opening/shutting control element 1640 and the pressurized canister 1510, the pressurized canister 1510 is shaken 432 and/or heated to a temperature in excess of 25° C., such as above 30, 35, 40, 45, 50, 55, or 60° C. for a period of time, such as in excess of 1, 2, 3, 4, 5, 10, or 20 minutes. For instance, for the case of a pressurized cheese product, the heating decreases the viscosity of the cheese to form at least a layer of liquid cheese product, which facilitates natural liquid-to-liquid movement of a liquid form of the amendment contents 1563 into the liquid/high viscosity zone 1540, which results in a more homogenized or evenly distributed content of the amendment contents 1563, such as the THC, in the liquid/high viscosity zone 1540. Optionally and preferably the heating step heats to a temperature above that of a typical liquid bath sterilization step of the resultant packaged product. As the optional temperature mixing step optionally and preferably exceeds temperature and time requirements of a typical liquid bath sterilization step, the heating step optionally replaces the sterilization step. Optionally and preferably, the heating step elevates temperatures of the amended product 114 to a temperature less than a temperature at which THC degrades, as described supra.

Still referring to FIGS. 15, 16A, and 16B, the pressurized canister 1510 is optionally any type of pressurized container, such as an aerosol container where the pressure is distributed with the canister in contact, interspersed into, and/or dissolved within the food product; a valve type container, where the pressurized gas is behind a valve and force the valve to move toward a dispensing valve forcing the food product out of the container when the valve is opened; and/or is a bag-in-can type canister.

Referring now to Table 4, two sequential methods are provided, the sequential methods corresponding to sequential action of the second to fourth column of Table 4, for amending and/or finalizing a food product in a pressurized container.

TABLE 4
Finalizing/Amending Product

| | | | Additional |
|---|---|---|---|
| | | Subsequent | Manufacturing |
| Case | Manufacturing Step | Manufacturing Step | Step |
| First Case | Open Pressure Seal | Add Product | Pressurize and |
| | | Constituent | Seal Product |
| Second Case | Open Pressure Seal | Add Product | Seal Product |
| | | Constituent while | |
| | | Increasing Pressure | |

Add THC to On-Site Prepared Formulation

Referring now to FIGS. 16 and 17, examples of the amend formulation 750 method of the process of amending the starting product 130 are provided. Generally, THC 210, a THC emulsion 530, and/or the additive 200 are optionally added to any food/beverage production, even if the food/beverage production step takes place at one location, such as a licensed THC facility. However, steps described herein aid in the production, such as in terms of enhanced homogenization of THC within a food product by adding the THC emulsion to an existing food/beverage formulation process and/or substituting in the THC emulsion 530 in place of THC 210 or a THC in oil in the formulation, as described supra. For clarity of presentation and without loss of generality, examples of amending a brownie preparation and amending a brite tank ready for canning or bottling are described, where the formulation of the brownie and/or the brite tank is amended to include the THC 210 and/or the additive 200 in the form of an emulsion.

### Example I

In a first example, a brownie formulation is amended. In the THC world, addition of THC to brownies is well known. However, the THC is in the form of raw/concentrated THC. Herein, amendment of a traditional formulation, such as that of a brownie, is described where the THC is added as an emulsion having specific properties, as described supra, that enhance suspension time in an aqueous solution and/or enhance homogeneity, as it is commonly known that traditional THC brownie recipes end up with a majority of the THC in a minority section of a batch of brownies.

Referring now to FIG. 17, in a process of adding THC to an on-site prepared formulation 1700, the THC emulsion 530 is added to a beverage 724; an edible 725, such as a food product, a cookie dough 1730, and/or a cream 1740, such as for packaging in a pressurized or non-pressurized whipped cream container; and/or is added to a product 726, such as a preroll or filling solution for a vape pen. A preroll, or pre-roll, is a joint prepared by a cannabis vendor, dispensary, or brand. Prerolls are consumer-ready and eliminate the hassle of grinding, rolling, and sealing cannabis flower into a joint. Prerolled joints are typically available in strain-specific flavors and vary in weight and potency.

### Example II

In a second example, beverage components, such as in a pre-packaging state in a brite tank, are amended with THC and/or a THC emulsion, where contents of the amended brite tank are subsequently canned or bottled In this case, optionally and preferably the original product is a premixed syrup concentrate, which is mixed with water in the brite tank. Here, the amendment process introduces THC into the syrup/beverage mix and/or the brite tank container, where a resultant THC amended brite tank mix is subsequently canned or bottled. Referring now to FIG. 18, a process of canning and/or bottling 1800 optionally and preferably mixes the THC emulsion 530 with constituents of a beverage in a brite tank 1820 and fills 1830 a can or bottle with the mixture/amended beverage. Optionally, the THC emulsion 530 is co-injected with constituents of the beverage in the brite tank 1820 in the process of filling 1830 a can or bottle to form the now amended beverage.

In the previous two examples, or more generally in any amendment process, the THC emulsion used in an amendment process of the original product optionally and preferably has a mean THC micelle diameter, excluding micelles of less than 20 nm diameter, in a range of: less than 300 nm, 200 to 1000 nm, 300 to 700 nm, 300 to 1000 nm, 400 to 700 nm, 500 to 1500 nm, and/or 1000 to 2500 nm, where the identified micelle diameters are optionally used for any formulation described herein. The amended formulation using a THC emulsion optionally and preferably has mean THC suspension viscosities of: 1 to 15 mPa sec, 5 to 30 mPa sec, 5 to 100 mPa sec, and/or 5 to 500 mPa sec. Optionally and preferably, the THC emulsion is passed through a nozzle of less than 1, 0.5, 0.1, or 0.01 mm using an applied pressure of greater than 150, 250, 500, 1,000, or 3000 bar to generate mean micelle emulsion diameters of less than 700, 500, 300, 200, or 100 nm.

Childproofing/Adult Use

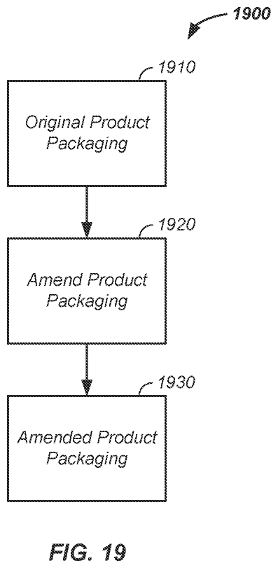

Referring now to FIGS. 19, 20, 21, 22(A-C), 23A, 23B, 24, 25, and 26 processes for protecting children from the effects of the THC and/or the nootropics are described. Generally, referring now to FIG. 19, a process of changing labeling 1800 of the original product 112 to represent the amended product 114 is described. The original product 112 may or may not have a label on it for sale. In cases where the original product 112 was labeled for sale without inclusion of THC, labeling of the original is optionally altered, changes, amended, and/or replaced to represent the amended product 114. For instance, the original product packaging 1910 is optionally and preferably amended 1920 to yield amended product packaging 1920. In some cases, the original product 112 is available for sale, but is shipped to the amendment facility already labeled to represent the amended product 114. Examples are used to further describe optional and preferably labeling changes and/or child protection devices.

### Example I

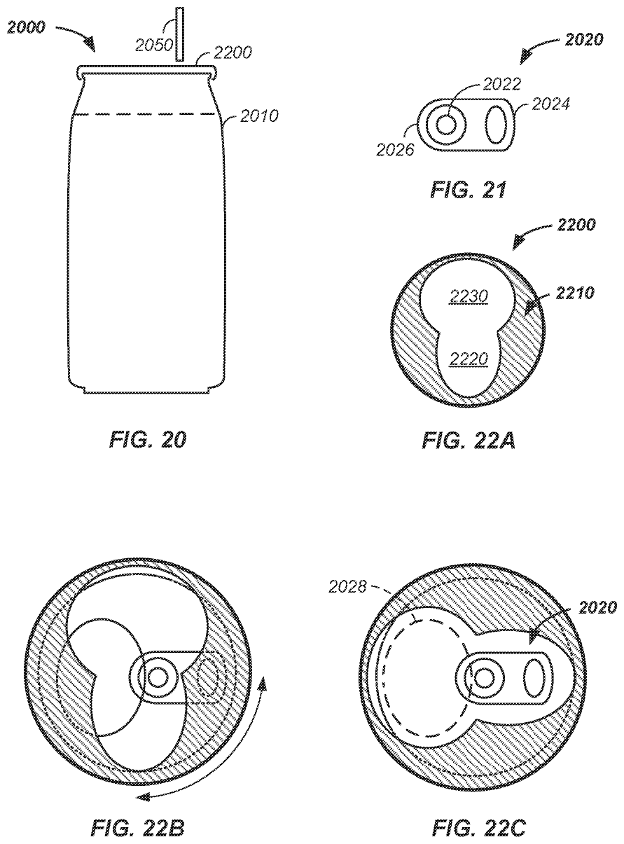

Referring now to FIGS. 20, 21, 22A, 22B, and 22C, a first example of child-proofing a THC containing can is provided. Referring now to FIG. 22, a rotatable can lid system 2000 is illustrated with a can 2010 and a rotatable cover 2200. Referring now to FIG. 21, the can 2010 and a can tab 2020 are illustrated. The can tab 2020 includes a pivot point 2022, a lever end 2024 operable by a user, and a pressure opening end 2026 that redirects the user's applied force downward to an openable portion 2028 of the can 2010. Referring now to FIG. 22A, the rotatable cover 2200 contains at least two accessible zones, an opener zone 2220 and a can opening zone 2230. As illustrated in FIG. 22B, at a first time, such as at time of manufacture and distribution, the rotatable cover 2200 prevents access to the lever end 2024 of the can tab 2020 and the openable portion of the can 2028. As illustrated in FIG. 22C, at a second time such at time of use by an adult consumer, after rotation of the rotatable cover 2200, the opener zone 2220 gives access to the lever end 2024 of the can tab 2020 and the can opening zone 2230 gives access to the openable portion 2028 of the can. Thus, rotation of the rotatable cover 2200 is required to access contents of the THC containing can. Optionally and preferably, the rotatable cover 2200 clips securely over a top of the can during production.

### Example II

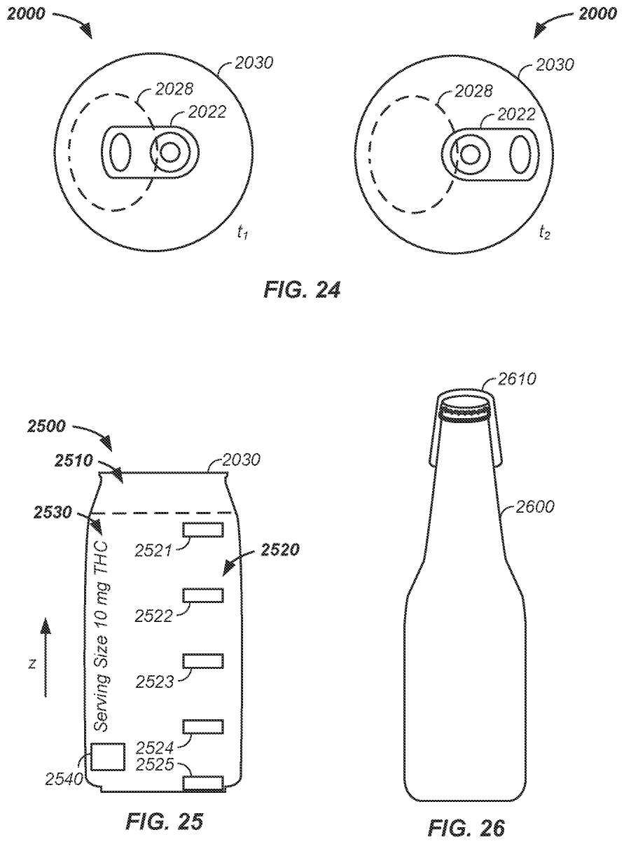

Referring now to FIG. 20 and FIG. 24, a second example of child-proofing a THC containing can is provided. As illustrated in FIG. 20, at time of production the tab 2020 is rotated around the pivot point 2022 relative to an operable opening position, such as either in original assembly or with a mechanical rotator 2050 used to spin the tab 2020. As illustrated in FIG. 24, at a first time, such as at time of sale to a consumer, the can tab 2020, if operated by a consumer, fails to apply a downward force on the openable portion 2028 of the can 2010. At a second time, such as after the consumer rotates the can tab 2020 around the pivot point 2022, the can tab 2020 is orientated in a traditional position and functions to open the can 2010 when the user levers up the can tab 2020 relative to a top 2030 of the can 2010. Optionally, the tab opener of a canned beverage is rotated from a non-opening position, such as rotated 180 degrees in the x/y-plane, to an opening position, where the z-axis aligns with gravity when the can is sitting upright. Thus, rotation of the can tab 2020 is required to access contents of the THC containing can.

Serving Sizes

Optionally, a beverage containing THC is packaged into a can with a resealable lid, such as provided by Sip N Shut (SNS Tech, Austin Tex.), Xolution (XOLUTION GmbH, Germany), and/or Heat Genie (Austin, Tex.). Optionally, the beverage container, such as holding a carbonated beverage, is marked with servings along a vertical axis of the container. For instance, if the legal limit per serving is 10 mg THC, then the can is marked at 10 mg intervals or 1 serving intervals along the vertical axis and the can is resealable between servings. Combined, the resealable container and the graphical serving size indicators allow a consumer to consume a known amount of the contents in the container and to consume a known amount of THC. For clarity of presentation and without limitation, two examples are provided of reclosable/recloseable/resealable containers, which are optionally and preferably additionally marked with 1, 2, 3, or more graphical serving size indicators.

### Example I

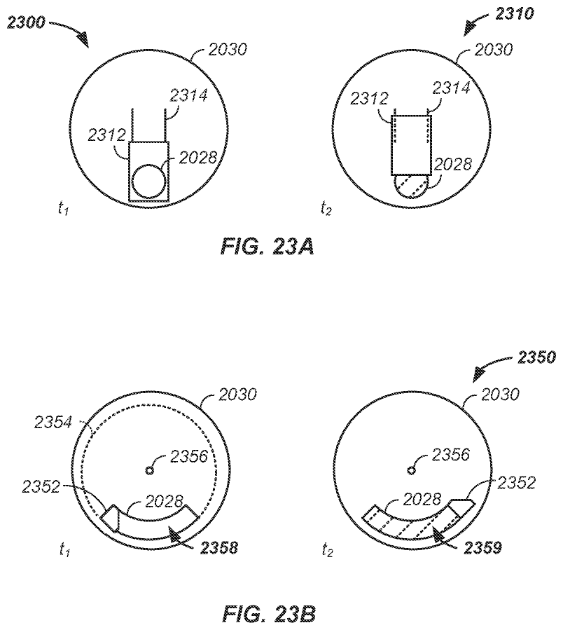

Referring now to FIG. 23A, a first example of a resealable lid 2300 on a container, such as an aluminum can, is illustrated. In this example, resealable closure, such as using a slideable tab 2312, also referred to as a slideable closure element, slides, such as along a guiding element 2314, from a first closed position 2028 to a second open position 2029. In the closed position 2028, such as at a first time, t₁, the contents of the container are sealed inside the container. In the open position 2029, such as at a second time, t₂, the contents of the container are held inside the container by gravity, but are allowed to pour out of the opening, such as when the container is upended. The slideable tab 2312 is optionally repositioned in the first position to reseal the remaining contents in the container. The guide element 2314 optionally and preferably includes a guide rail that limits movement of the slideable tab along an axis aligned with the guide rail and/or a mechanical stop that limits an extent of movement of the slideable tab, such as along the guide rail or along an axis passing through an opening in the lid of the can. By repeating the process while paying attention to the level of the beverage in the container relative to graphical serving indicators on the container, further described infra, the consumer may control intake/served dosage of the THC, such as to 1, 2, or more servings or fractions thereof.

### Example II

Referring now to FIG. 23B, a second example of a resealable lid 2300 on a container, such as an aluminum can, is illustrated. In this example, a rotatable tab 2352 rotates, such as around a central point and/or guide pin 2356, from a first closed position 2028 to a second open position 2029. In the closed position 2028, such as at a first time, 6, the contents of the container are sealed inside the container. In the open position 2029, such as at a second time, t₂, the contents of the container are held inside the container by gravity, but are allowed to pour out of the opening, such as when the container is upended. The rotatable tab 2352 is optionally and preferably connected to a rotatable disc 2354, where the rotatable disc 2354 includes at least one open section, such as a gap in the disc 2354, that is rotated between a position of an opening of the container to form an opening 2359 and at least one closed section, such as aluminum, covering the opening 2358 to form a closure 2358 of an opening in the resealable lid. For clarity of presentation herein, the rotatable disc 2354 is optionally referred to as a slideable disk, where the disk 2354 slides, relative to the top of the container, around the guide pin 2356, with or without a sealing element between the rotatable disc 2354 and the top of the container. The rotatable tab 2352, that helps rotate the disc 2354 by human applied force, is optionally repositioned in the first position to reseal the remaining contents in the container. Again, by repeating the process while paying attention to the level of the beverage in the container relative to graphical serving indicators, further described infra, the consumer may control dosage of the THC, such as to 1, 2, or more servings or fractions thereof.

In these examples, the beverage is optionally and preferably a soda, a coffee, a tea, a nootropic containing solution, an alcoholic drink, and/or a drink that contains ethanol, such as at a concentration exceeding 0.5, 1, 2, 3, 5, 10, 15, or 20 percent ethanol by volume, where the beverage optionally contains any amount of Cannabis, a Cannabis product, CBD, and/or THC.

In both examples, the element grabbed by a user to move alternatingly unseal/seal the container and/or a moveable element used to seal/unseal the container optionally and preferably remain affixed to the container when in a sealed position and when in an open position.

In both examples, the resealable element, such as the slideable tab 2312 and/or the rotatable disk 2354 optionally and preferably slides and/or rotates on a plane within ten degrees of parallel to a top of the container 2030, such as a lid of the container, where the top of the container 2030 and/or the lid is within ten degrees of horizontal when the container is positioned in an upright orientation with a vertical axis of the container aligned with gravity.

Adult Use Labeling

Referring now to FIG. 25, the can 2010 optionally and preferably contains an adult use labeling zone 2510 near the top 2030 of the can. For instance, optional packaging labeling to represent the amended product 114 includes one or more of: adding a “for adult use only” label, color coding a section of the label, where the color coding indicates to a trained consumer that the product is for adult use, color coding a particular section of a container, such as the labeling zone 2510, a top ½ inch, a top ¾ inch, and/or a top 1 inch plus-minus ⅛ inch sections of the listed sections, color coding a section of the packaging, such as the labeling zone 2510 and/or an upper portion of a beverage container, such as with a green, bright green, orange, bright orange, yellow, or bright yellow label, where the color coded warning section is optionally labeled with wording indicating that the product is for adult use, contains THC, and/or a combination of the above. Referring now to FIG. 26, optionally, additional packaging, such as a seal 2610, is added to the original packaging, such as to a bottle 2600, as a child-proof opening constraint, an adult-use only labeled wrap about at least an opening portion of a can/bottle, and/or a color coded label as described supra.

Still referring to FIG. 25, the can 2010 optionally and preferably includes one or more markings related to serving size, such as a serving size of yielding an amount of THC. For instance, a text label indicates a serving size 2530 of THC, such as “Serving size 10 mg THC”, where the serving size is optionally and preferably 1, 2, 5, 10, 15, 20, 25, or 50 mg THC and/or is less than or equal to 500, 200, 100, 50, 40, 30, 20, 15, or 10 mg THC. Optionally and preferably, the can 2010, bottle 2600, or container contains indexed graphical markings 2520 indicating sample size, such as one serving 2521, two servings 2522, three servings 2523, four servings 2524, five servings 2525, or n servings, where n is a positive integer of 1, 2, 3, 4, 5 or more. Optionally, the graphical markings 2520 are windows through a label, where the solid or liquid inside is viewed through a transparent portion of the container. For example, a soda or beverage is marked with servings along a vertical axis, z-axis, and/or an axis aligned with gravity, where the amount of soda remaining and associated marking/window indicates how many servings have been ingested, poured, and/or removed from the container. Optionally, the graphical markings 2520 are graphical symbols representing an amount of THC consumed.

Consumer Labeling

A THC package, container, can, bottle, and/or jar is optionally labeled in a manner to convey information about the Cannabis, Cannabis derived molecule, and/or THC in the package. Still referring to FIG. 25, optionally and preferably a two-dimensional barcode and/or a consumer QR (quick response) code 2540 is placed on the packaging/labeling containing the THC, where the QR code provides a link to specific information about the marijuana product. For instance, the consumer QR code provides one or more of: a marijuana strain contained in the package; a certification of an extraction process, such as a carbon dioxide extraction versus an alcohol extraction step; a lab test result indicating one or more impurities, such as a concentration of a heavy metal and/or presence and/or concentration of agrochemicals; a certification of a concentration/dosage of THC in the package and/or in a serving in the package; a distribution chain including at least a point of intended sale; a chain of custody trail of the THC from seed to sale; a location of grow; a date of harvest; and/or an expiration date. Optionally, the QR code is specific to the individual package available for sale as opposed to a QR code that is used for all products shipped in a generic package. The final QR code, which is optionally a unique identifier (UID) thus aids transparency of the marijuana product history, age, and/or purity to the consumer at the point of sale.

Herein, a QR code, which is now terminology in common usage, is also an abbreviation for a quick response code. Generally, a QR code is matrix barcode, such as a two-dimensional barcode, which is a machine readable optical label. The QR code optionally and preferably contains information, such as about the labeled item, location, position, and/or product. For instance, the QR code contains data about the THC product, a history of the THC product, legal information, tracking information, and optionally and preferably points to a website that equivalent information in a human readable form. The QR code label is optionally of any geometry, such as not being limited to rectangular. The QR code is optionally color coded. However, current QR codes are high contrast colors, such as black and white. The QR code is optionally unique to a specific item. For instance, if 2, 10, 100, 1000, or more items come off of an assembly line, there are optionally a set of 2, 10, 100, 1000 or more unique QR codes with a unique QR code per one unique item. For example, a first THC portion and a second THC portion that are otherwise indistinguishable optionally have a first QR code and a second QR code, which allows individual tracking of each of the THC portions downstream, such as in addition to products, distribution, and/or sale.

For instance, the QR code associated with a product, referred to herein as an end user THC QR tag, such as printed on a THC product labeled for sale to a consumer/purchaser, is optionally and preferably associated with/linked with, one or more of:
a location of harvest of a plant containing THC in the THC product and grow area code, such as via a grow area QR code and/or a grow area RFID code;
a date of harvest/a date of trimming of the plant containing THC in the THC product, such as via a trim code, such as a trim QR code and/or a trim RFID code;
a THC strain of THC in the THC product, such as indica, sativa, or hybrid;
a type of THC present in the cannabis plant, such as: THCA, THCV, Delta-8 THC, and/or Delta-9 THC;
a type of extraction process used to extract the THC, such as a CO2 extraction, salt extraction, altered pressure extraction, and/or a distillation extraction, and is optionally and preferably linked to an extraction code, such as an extraction QR code and/or an extraction RFID code;
a THC type in the THC product;
a date of manufacture of the THC product;
a location of manufacture of the THC product, such as a product formation QR code and/or a product formation RFID code;
quantity of THC in the THC product;
a distribution chain of THC in the THC product, such as a distribution QR code and/or a distribution RFID code;
a point of sale of the THC product;
a tax stamp associated with the THC product; and
purchaser information of a purchaser of the THC product.

Said another way, the end user THC QR tag optionally and preferably contains information linked with any QR tag and/or information linked with any RFID tag for any process and/or step leading to the final THC containing product or product. Thus, the consumer/purchaser may scan the final end user THC QR tag and obtain information on any recorded seed-to-sale information, such as grow location, strain, harvest date, extraction type, date of manufacture of the product in hand, and/or a distribution chain of the product in hand. For instance, information in and/or associated with any of: (1) a first QR code and/or a first set of one or more RFID codes, such as on (QR) and/or associated with (RFID) a wrap-around tag on an individual marijuana plant contains first information about the THC in the final product; (2) a second QR code and/or a second set of one or more RFID codes, such as on (QR) and/or associated with (RFID) a temporary tag, such as on a first process step container used in extraction and/or formation of the THC in the final product; and/or (3) a third QR code and/or a third set of one or more RFID codes, such as on (QR) and/or associated with (RFID) a sticker tag, such as on a second process step container used in formation and/or distribution of the final THC product is optionally and preferably included in a link linked to by the end user THC QR tag, such as placed as a permanent label on an end-label on the THC product for sale to the consumer, optionally and preferably along with any warning described herein, next to product advertising/labeling on the end THC product for sale to the consumer.

Optionally, a QR code includes a link to any required information, such as a transactional stamp, which is also referred to as a secured data identifier or a report that contains required information to ensure accuracy of reported information; a time, a date, and/or a location of a production, delivery, and/or sale; an operator's identification; an agent number uploading marijuana related information; a name of a marijuana facility; a programming note stamp; a THC related report; a chain of custody of the THC; a history of uploads to the linked information; and/or a history of deletions of information from the linked information. The report and/or the transactional stamp is optionally and preferably linked to output of one or more video camera recorders that cover rooms within the THC facility; data included on an RFID tag; and/or employee tracing information.

Emulsion Formation

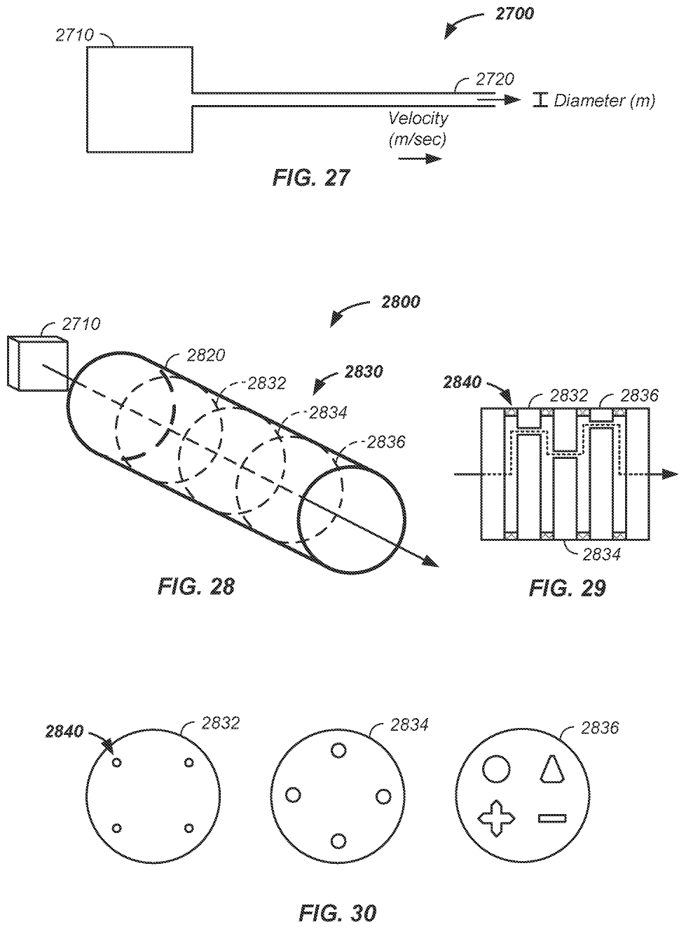

Referring now to FIGS. 27-30, examples of the high-pressure homogenizer 668 are provided.

### Example I

In a first example, a first high pressure emulsifier 2700 is illustrated. Generally, a pump 2710 pumps an emulsion, such as prepared by the mixer 662 through a tube/open ended container 2720, such as a capillary tube. One definition of a shear force is velocity over distance, such as velocity through the tube 2720, such as in m/sec, with a distance between the walls of the tube, such as in meters, where the velocity in m/sec over distance in meters reduces to 1/sec or inverse seconds. Similarly, velocity through a rotator stator is the velocity of flow between rotator and the stator and the distance is the distance between the rotator and the stator. Thus, labels here of inverse second are optionally labeled as velocity/distance or meters/sec divided by meters. Shear forces in the tube break down the emulsion particles into smaller volumes/micelles. For example, the rotator-stator mixer forms micelles in a range of 1000 to 2500 nm in diameter, which are broken down by high shear forces in the high pressure emulsifier to mean diameters of less than 700, 500, 300, or 200 nm, as described supra. The shear forces in the first high pressure emulsifier are optionally and preferably greater than 100,000, 1,000,000, 5,000,000, or 10,000,000 inverse seconds. For a straight tube, the shear forces are provide by equation 1,

ΔP=(v·s·l)/(4·1000·d) (eq. 1)

where P=pressure in Pascals, v=viscosity in mPa·sec, s=shear rate in inverse seconds, l=length in millimeters, and d=diameter in millimeters.

### Example II

In a second example, a second high pressure emulsifier 2800 is illustrated. In this example, the high pressure pump 2710, such as a greater than 2000, 3000, 4000, 5000, 10,000, 20,000, or 30,000 p.s.i. pump, forces the emulsion, such as THC, water, and lecithin, through a container 2830, where the container contains a set of emulsion/shear plates 2830 separated by a set of spacers 2840. For instance, the high pressure pump 2710 forces the emulsion through holes/openings in a first shear plate 2832, then through a second shear plate 2834, then through a third shear plate 2836, . . . , and finally through an n^{th} shear plate, where n is a positive integer of greater than 0, 1, 2, 3, 4, 5, 6, 8, 10, 15, 20, 40, 80, or 100. Optionally and preferably, holes in subsequent shear plates are not aligned, resultant in a tortuous path of the emulsion both between plates and through plates where: (1) the close distances between plates, such as less than 50, 10, 5, 2, 1, 0.5, 0.1, 0.01, or 0.001 mm, result in shear forces between plates and/or (2) narrow mean openings/holes through each shear plate, such as less than 20, 10, 5, 2, 1, 0.5, 0.1, 0.01, or 0.001 mm, result in shear forces through plates to yield a preferred shear force of the system on the emulsion, which yields the preferred stable and/or transparent emulsion with mean particle sizes of less than 1000, 750, 500, 300, 200, or 100 nm. As illustrated, the openings 2840/holes through the shear plates are optionally of any size and/or geometric shape. However, larger perimeter to diameter ratios are preferred due to an increase in exerted shear forces, such as produced by a circle, triangle, rectangle, polygon, or the illustrated cross shape, which comprising indentations (tabs) into and cutouts around a circle shape.

THC Facility

Optionally and preferably, any machinery in a THC licensed facility is configured to operate only after a bar code is read on-site by a bar code reader, the bar code is compared with a look-up table of acceptable bar codes, such as linked to THC licensed/approved operators, and an approval is sent to the machinery, such as a sprayer, dropper, injector, and/or packaging system, such as on as assembly line.

### Optional Embodiments

A number of additional methods of manufacture and/or uses of the emulsions and/or methods and apparatus for dispensing THC to a user are provided by way of examples herein.

### Example I

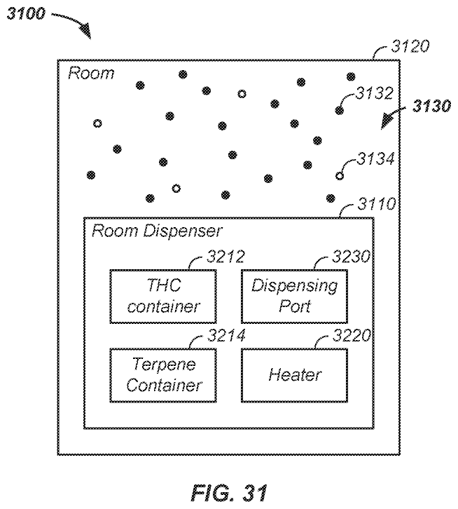

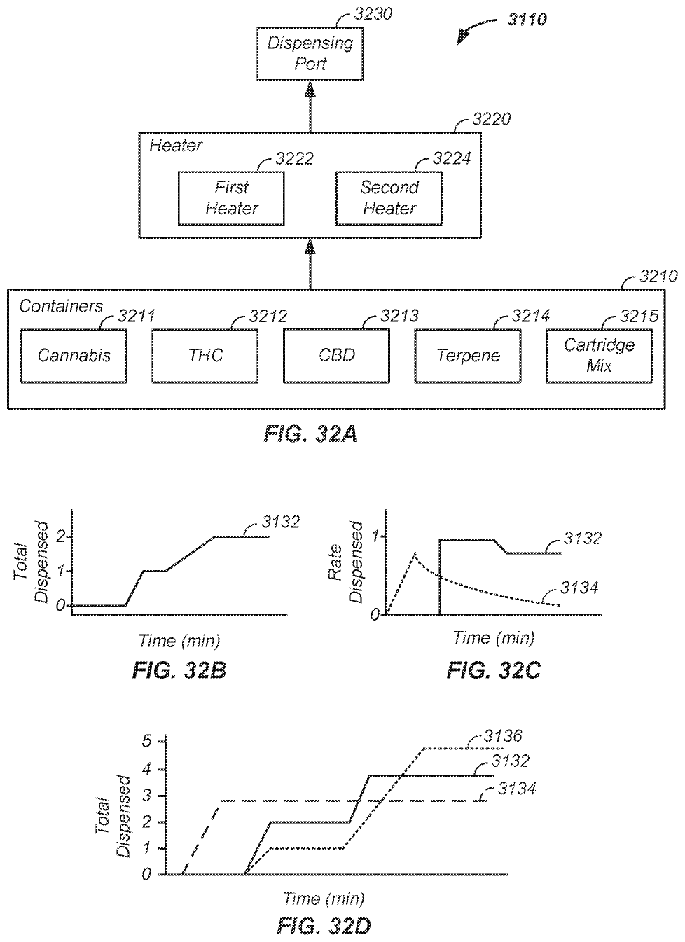

Referring now to FIG. 31, in a first room dispensing 3100 example, THC is released via use of a room dispenser 3110 into a room 3120 and/or the atmosphere, such as in the form of a gas, a liquid droplet, and/or with THC bound to a carrier, such as in an emulsion. The room dispenser is optionally a fogger, a nebulizer, and/or a diffuser. For clarity of presentation and without loss of generality, in one case, the THC is released/dispensed from a fogger. Herein, a fogger releases the THC into the room 3120 in a gas form of THC or the THC solid/liquid is broken into a liquid/vapor like state, such as with a diffuser, a nebulizer, and/or by way of treating the THC with ultrasonic energy. In another case, an aroma lamp, nebulizer, and/or a diffuser is used to diffuse oils, such as THC, into the air by heating it and/or by nebulizing the THC using compressed air or ultrasonics. Optionally, the nebulizer is a drug/THC delivery device used to administer the drug/THC in the form of a mist inhaled into the lungs. In any of these methods, oxygen, a compressed gas, compressed air, heat, and/or ultrasonic power is used to break up the THC into a suspension of small aerosol droplets that are subsequently inhaled, such as through a mouthpiece. Referring now to FIG. 31 and FIG. 32, generally, in the room dispenser 3110, optionally THC is held in a THC container 3212 with or without terpenes and/or terpenes are held in a terpene container 3214. The THC/terpenes are delivered to an area heated by one or more heaters 3110 and/or nebulizer/diffuser component and the resulting gas/liquid droplet/emulsion is dispensed through an dispensing port 3230 and/or outlet into the room 3120. As illustrated, dispensed elements 3130 include an airborne version of THC 3132 and/or an airborne version of terpene 3134.

Similarly, an inhaler, puffer pump, or sprayer is optionally used to dispense the THC directly into the respiratory system, such as the lungs or nose via a mouthpiece into the mouth. The inhaler is optional of a metered-dose type, a dry powder inhaler, a soft mist inhaler, and/or a nebulizer. The inhaler optionally uses pump force action and/or a compressed gas, such as carbon dioxide or compressed air, to expel small amounts of the THC from the inhaler container.

### Example II

Referring now to FIG. 32(A-D), in a second example, the THC is optionally dispensed into the atmosphere/room 3120 from a fogger/mister/dispensing apparatus/room dispenser 3110 with the aid of a heating element and/or a heater 3116. The heater 3116 is optionally controlled with a temperature controller, which controls the dispensed substance at a set or programmable temperature as further described infra. For instance, substance are brought to a liquid/gas phase transition temperature and/or smoke point by one or more programmable heaters. Optionally and preferably, a heating element, such as a ceramic heater, heats the oil/THC/dispensed substance, such as near a dispensing area and/or from a wick of a refill cartridge/refill canister causing the oil to flow more readily as a fluid and/or to form small airborne droplets/vaporize/atomize/go into a gas phase.

Optionally and preferably, in any of systems described herein, the THC is elevated in temperature above 20° C. and preferably above 40, 50, 60, 70, 75, or 80° C. and below 120, 150, or 200° C. with a heater and optionally a temperature controller, which enhances the amount and/or rate of forming the droplets/particles/gas. Once a gas state of the THC is reached, further heating is optionally and preferably reduced/eliminated to avoid burning the THC.

Referring now to FIG. 32A, one or more containers 3210 hold together or separately cannabis 3211, THC 3212, CBD 3213, a terpene 3214, a cartridge mix 3215 of constituents, and/or any of the substances described herein. The heater 3220 is optionally one or more heaters, such as a first heater 3222 and a second heater 3224, which are used to heat any of the substances described herein to any temperature at any rate at any time. The heated components are optionally and preferably released from a heating system and/or the room dispenser 3110 through one or more dispensing ports 3230, such as in a form of a droplet, mist, aerosol, or the like.

Referring now to FIG. 32B, the THC is optionally released at different times and/or at different rates in an airborne form of THC 3132. As illustrated, THC is released at a first rate over a first time period and then after a pause released at a second rate over a second time period. Referring now to FIG. 32C, an example of the airborne form of THC 3132 and an airborne form of a first terpene 3134 being dispensed at different rates from the room dispenser 3110 is provided, where the different rates are constant, accelerating, and/or decelerating, where the rates for dispensing each substance optionally differ for the two constituents as delivery times and rates from the containers 3210 optionally varies as do heating times for the heater 3220 associated with each substance. Optionally, many terpene containers are used and individual terpenes are dispensed at independent rates, such as illustrated in FIG. 32D where a second terpene 3136 is dispensed at rates/dosages differing from the first terpene. More generally, THC or any of the constituents described herein are optionally released from the room dispenser 3110 into the room 3120 at any number of times, where the times are optionally different for each of the constituents, and/or at any number of rates.

THC is optionally released into the atmosphere, such as to permeate a room at a programmed rate, such as a rate that increases with time, is constant with time, and/or decreases with time.

Optionally and preferably, in any of the systems described herein, a secondary component is heated to a secondary temperature. For instance, a terpene is a secondary component. Optionally and preferably, a second heater, or a first heater at a second time, is used to heat the terpene to a temperature that phase changes the terpene from a solid and/or a liquid into a gas. For instance, referring again to Table 1, the terpene pinene has a 155° C. boiling point. Individual terpenes are optionally mixed together, such as in a single container or infused into a preroll paper used to wrap prerolls, or are individually stored until heated in separate containers. In the first case, the temperature is optionally ramped upward to release a first terpene, then a second, then a third or is flash heated/heated rapidly to release a subset or all of the terpenes at once. In the second case, each terpene is heated to a boiling point separately or in a mix delivered to a heating area heated by the heater.

Still referring to FIGS. 32(A-D), optional components emitted by the room dispenses 3110 include: oxygen, phenols, or any component having a smell. For instance, at a concert during first period, a darker lighting period, a darker music period, and/or a more dramatic sound period, leather like odors and/or an indica THC strain is released and during a second time period, second odors and/or a second THC strain is released.

### Example III

In a third example, a nootropic, such as any of the nootropics described above, CBD, and/or CBG, is added to an emulsion with or without THC, where the emulsion has any of the properties described supra. The pH of the emulsion is optionally and preferably maintained at a pH of 2, 3, 4, 4.5, 5, 5.5, 6, 6.5, 7, 7.5, 8, 9, 10, 11, or 12±0.8, 0.5, 0.3, 0.02, or 0.1 pH units. The THC, CBD, CBG, nootropic, mushroom emulsion is optionally sold as an additive to a manufactured product and/or is sold to an end user where the end user adds the emulsion, optionally referred to as nano fuel, to any food and/or beverage product made in the home or purchased commercially, such as in a THC illegal zone.

### Example IV

In a fourth example, a THC, CBD, CBG, nootropic, and/or mushroom emulsion, such as any of the emulsions described herein, is manufactured in a first zone where THC production is legal and used to modify a food and/or a beverage in a second zone, where it is illegal to purchase and/or manufacture a THC product, such as at a THC concentration exceeding 0.3% by mass or any of 0.1, 0.2, 0.3, 0.4, or 0.5% by volume, weight, or mass. Optionally, the THC containing emulsion is districted from the first zone to the second zone through a legal dispensary.

### Example V

In a fifth example, THC, with or without terpenes, as a liquid form of THC and/or as an emulsion containing THC is placed onto and/or permeates into an incense stick. Notably, as the incense stick heats the THC and/or terpenes present are heated to temperatures sufficient to phase change the THC/terpenes into a gas phase, which then permeates the air space and/or enters the lungs via the mouth and/or nose.

### Example VI

In a sixth example, a candy including sugar, flavoring, and an embedded gas, such as a carbonated candy and/or “Pop Rocks”, owned by Zeta Espacial SA (Barcelona, Spain) is modified with a THC, CBD, CBG, a nootropic, and/or a nootropic oil containing emulsion, where the candy is a carrier of the oil(s). Optionally, the candy is made by dissolving one or more sugars in water; evaporating the water to a content of less than 10, 5, 4, 3, 2, or 1 percent by mass in the candy and greater than 0, 1, or 1.5 percent by mass; and pressuring the candy at pressures exceeding 1.5, 2, 5, 10, 25, 40, 50, or 75 atm, optionally and preferably while stirring the candy, with a gas, such as air, carbon dioxide, nitrogen, and/or nitrous oxide where the carbon dioxide, nitrogen, and/or the nitrous oxide are present at concentrations at least 1.5, 2, 5, 10, or 20 times that found in nature and optionally at ratios relative to each other exceeding 1:1, 2:1, 5:1, or 10:1. Optionally and preferably, the dissolved gas bubbles have a mean diameter, excluding those under 10 micrometers, of 100±50, 150±25, 175±25, 200±25, 250±50, 300±50, 350±50, 400±50, or 500±100 micrometers. When pressure on the, optionally and preferably cooled and solidified, candy is released, the candy shatters and/or is crushed into pieces ranging in mean size, excluding those less than 0.009 inches, from 0.01 to 0.25 inches in largest cross-sectional size. Optionally, the candy is referred to as cosmic fizz.

### Example VII

In a seventh example, the THC emulsion is added to a caffeinated energy drink/energy shot, which is optionally carbonated. The caffeine in the drink is optionally and preferably greater than 50, 100, 150, 180, 190, 200, 210, or 220 mg and less than 300, 400, or 500 mg, where the caffeine is delivered in a total volume of the energy drink/energy shot of less than 20, 15, 12.2, 12, 10, 8, 6, 4, 2, or 1 ounces. Optionally any Cannabis component is used in the energy drink/energy shot. Optional ingredients in an energy drink/energy shot include, but are not limited to: caffeine, sugar, glucose, high fructose corn syrup, maltose, galactose, sucrose, nitrous oxide, such as in a form of a partially dissolved gas in a pressurized contain, vitamin B6, folic acid, vitamin B12, sodium, taurine, glucuronolactone, malic acid, N-Acetyl L-tyrosine, L-phenylalanine, citicoline and/or the psychoactive dopamine precursor amino acids tyrosine and phenylalanine.

### Example VIII

In an eighth example, the amount of THC per serving and/or per container is optionally and preferably greater than 0.5, 1, 2, 3, 4, 5, 6, 7, 8, 9, 10, 15, 25, 50, 75, 100, or 200 mg and less than 2000, 1000, 500, or 250 mg.

### Example IX

In a ninth example, the THC emulsion and/or any drink or emulsion described herein is optionally flavorless or is flavored with natural and/or artificial flavors to any flavor, such as vanilla, coffee, berry, grape, watermelon, fruit punch, and the like.

### Example X

In a tenth example, the THC emulsion, with or without additives, is packaged in a glass and/or plastic container/vial with or without an eyedropper and the THC emulsion is delivered to the body sublingually, such as by placing one or more drops of the THC emulsion under the tongue. The eyedropper is optionally marked in intervals of THC dosage, such as 5, 10, 20, or 50 mg and/or with a linear range of 5, 10, 15, 20, . . . 50 or 100 mg THC or 10, 20, 30, 40, . . . 100, 200 mg THC or any subrange therein.

### Example XI

In an eleventh example, an app or computer program is used to track and/or suggest an amount/type of THC or the like for a given activity for a body type, usage history level, and/or an individual. For instance a 250 pound man might require more THC than a 150 pound man. Other optional parameters include: age, gender, BMI, height, weight, normal daily usage, event (workout, relaxation, high), and/or tolerance.

### Example XII

In an twelfth example, a carbonated beverage is produced that contains THC in the form of an emulsion. Optionally and preferably, at least a portion of the carbon dioxide used to carbonate the beverage is replaced with nitrous oxide, thus forming, what is termed herein, a nitrous drink. Nitrous oxide is also known as dinitrogen monoxide, N₂O, and/or “laughing gas”. Nitrous oxide is used to achieve an analgesic, such as by huffing, which is often referred to as a narcotic high. A nitrous drink is not to be confused with a nitro drink where nitrogen is added to the drink.

Herein, a nitrous drink contains at least double the concentration of nitrous oxide as found in the atmosphere and optionally contains at least one other gas, such as air, hydrogen, helium, nitrogen, carbon dioxide, and/or argon. Optionally helium is used in place of any of the nitrous oxide concentrations/pressures described herein.

During manufacture, nitrous oxide is optionally and preferably combined with a drink/drink mixture to be canned/bottled/sealed, such as in any size brite tank, such as greater than 1, 50, 150, 500, or 1000 gallons. The brite tank is optionally and preferably used as a reservoir for filling, bottling, or canning a beverage, such as the nitrous drink. Optionally and preferably the brite tank is pressurized with the nitrous oxide and optional secondary/additional gases. The pressure of the gases in a headspace of the drink are optionally greater than 5 or 10 psi and preferably within 10 psi of any of 20, 30, 40, 50, 60, or 70 psi at temperatures ranging from 40 to 100° F., such as in a standard aluminum can or within 25 psi of any of 50, 75, 100, 125, 150, 175, 200, or 225 psi in a canister rated for higher pressure, where the sealed pressure relates to foaming of the nitrous drink as described infra.

Optional and preferable foaming of the nitrous drink is described. Initially, the nitrous oxide is at least partially dissolved in the drink with the THC emulsion. Optionally and preferably, the emulsion and/or the THC emulsion contains an emulsifier, such as lecithin, milk, cream, an emulgent, egg yolk, a sodium phosphate, sodium stearoyl lactylate, soy lecithin, a Pickering stabilization, a diacetyl tartaric acid ester of monoglyceride (DATEM), PolyGlycerol Ester (PGE), PG Ester (PGME), MDGs, stearoyl lactylates, sorbitan esters, polyglycerol esters, sucrose esters, agar, albumin, alginates, casein, glycerol monostearate, gums, Irish moss, tragacanth, sodium lauryl sulfate, sodium dioctyl sulfosuccinate, and/or polymers known as the Spans and Tweens. Said again, the nitrous oxide optionally permeates the drink, emulsifier, and/or milk product/milk substitute/dairy product substitute. When the pressure in the nitrous drink is released, such as when the can/bottle is opened, the internal nitrous oxide in the liquid drink expands and foams/whips the emulsifier/cream/milk into a foam or froth, where the froth contains the nitrous oxide and/or the THC and where the nitrous oxide leads to a potential analgesic high, which complements the inhalation/ingestion of the THC, which is optionally supplemented with the B12, caffeine, and/or sugar rush of the drink, where the nitrous oxide leads to an initial high followed by affects of the other drink constituents. Thus, the nitrous oxide drink optionally and preferably generates a nitrous oxide head/foam on the drink when opened and/or poured.

Optionally and preferably, the nitrous oxide comprises at least 5, 20, 50, 75, 80, 85, 90, 95, 96, 97, 98, or 99% of the dissolved gases in the drink by mass. The nitrous oxide is optionally added to the drink via pressurization of the brite tank with the nitrous oxide/nitrous oxide mix gas and/or is placed into each can in the form of a solid or liquid, which phase changes into nitrous oxide gas inside the now sealed can. The amount of nitrous oxide per milliliter of drink is expressed here in terms of a twelve ounce soda, but is readily scaled to any size drink. The amount of nitrous oxide in a twelve ounce soda is preferably greater than 0.01, 0.1, 1, 2, 5, 10, 50, or 100 g and preferably in a range of 0.01 to less than 2, 3, 4, 5, or 10 g per can.

### Example XIII

In an thirteenth example, alcohol, such as ethanol, is added to THC, where the THC is an oil and/or an emulsion. Ethanol dissolves THC, which brings a very viscous pure form of THC, such as THC at greater than 50, 60, 70, 80, or 90% purity, into a less viscous phase, such as having a viscosity of less than 100, 25, 10, 5, or 2 newton-second per square meter (N-sec/m²) or Pascal-second (Pa-sec). The inventors have determined that the effect of ethanol on THC is to multiply the effective concentration of THC by greater than 2, 3, or 4 times. For instance, 50 mg of THC in ethanol results in an effective concentration of THC of 50*4 or 200 mg THC. Optionally and preferably, the THC/ethanol mixture, solution, tincture, and/or emulsion is added to a food, beverage, and/or product, such as to a preroll of Cannabis or containing THC, a gummy, a carbonated candy, and/or any of the products described herein. The ratio of ethanol/THC in the mixture, solution, tincture, and/or emulsion is optionally and preferably greater than or less than 0.001:1, 0.01:1; 1:1 2:1, 5:1, 10:1, 100:1 or 1000:1 by mass at time of formation of the mixture, solution, tincture, and/or emulsion. Optionally and preferably, the concentration of ethanol in the final product is less than 75, 50, 25, 10, 5, 4, 3, 2, 1, 0.05, 0.025, 0.01, or 0.001 percent by mass. Benefits of a lower concentration of ethanol is not being regulated as an alcohol products by a government body while still triggering the above described enhancement of effective THC dosage. Ethanol is optionally added to THC in any of the embodiments/examples described herein. Ethanol is optionally added to any food, beverage, product at a rate of less than 50, 25, 10, or 5 g per serving/unit of consumption.

### Example XIV

In a fourteenth example, ethanol is used to dissolve THC, such as a THC at greater than 50, 60, 70, 80, or 90% purity, into a less viscous phase, where the less viscous phase is easier to handle, such as by forcing through an emulsifier system, a tube with a pump, stirring, and/or mixing, such as in a manufacturing process of a food/drink/product.

### Example XV

In a fifteenth example, butter and/or a fat and/or a hydrophilic carrier is used to dissolve/carry THC in a product. For example, the THC/fat solution is added to popcorn kernels, oil, and/or flavorings packaged in a heavy gauge aluminum or microwave popcorn bag, such as in a Jiffy Pop® (Chicago, Ill.) like product, that is optionally and/preferably subjected to heat/heating wavelengths and popped prior to ingestion.

Emulsion

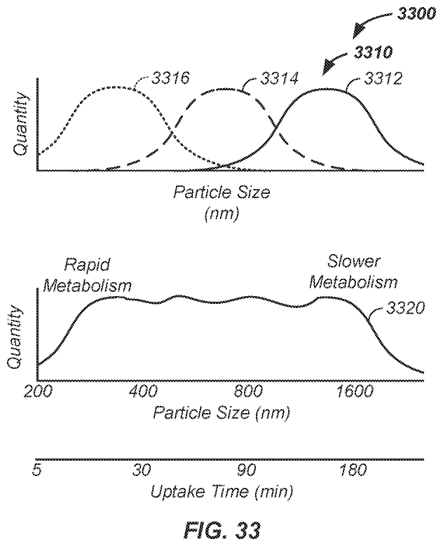

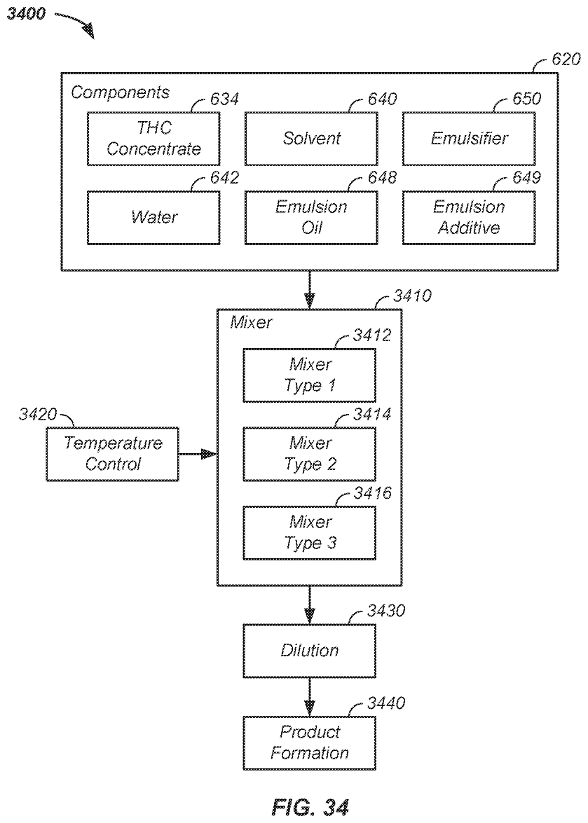

Referring now to FIGS. 33-36, THC emulsions are further described. Again, as described supra, herein a THC emulsion contains any component of Cannabis with or without THC and optionally contains any additive, such as a nootropic.

Referring now to FIG. 33, a THC emulsion with multiple particle sizes 3300 is described. Generally, the inventors have determined that smaller particle sizes of THC/oil droplets in an emulsion, such as a micelle or lipid bilayer cell are taken up faster in the body and result in an earlier high. For example, a micelle of THC in water with a mean diameter, herein referred to as a particle size, of less than 200, 300, or 400 nm is results in a faster uptake and a faster high, such as a high in less than 5, 10, or 15 minutes, than a micelle in water with a mean diameter of greater than 1000, 1500, or 2000 nm, which results in a high in greater than 20 or 30 minutes. Similarly, the inventors have determined that by mixing particle sizes, a prolonged/time-release high is obtained. As illustrated, a THC emulsion/particle size is generated by a mixing method 3310. Three mixing methods are used in parallel and/or in series to generate three particle size distributions that are subsequently mixed to form a long/time release emulsion 3320, such as optionally having a more or less even distribution of particle sizes, such as peak concentrations or peak concentration distributions at varying particle sizes that differ from a mean concentration by less than 100, 50, 10, 5, 4, 3, or 2 times. The long-time release emulsion optionally and preferably has a longer release time and/or a broader particle size distribution than mixing with one mixing technique, such as for a fixed period of time or over a fixed number of passes through the mixer. Examples of a long-time release emulsion include: more particles of less than 400 nm than greater than any of 400, 700, or 1000 nm; more particles of less than 700 nm than greater than any of 1000, 1500, or 2000 nm; at least one particle of less than and/or at 400, 500, or 600 nm and greater than 20 nm for every 1, 5, or 10 particles greater than and/or at 700, 1000, or 1500 nm; at least two particles at any one or more of 200, 300, 400, 500, 600, or 700 for every one particle at any one or more of 800, 900, 1000, 1250, 1500, 1750, 2000, 2500, or 3000 nm; at least one particle in a range of at least 20 and less than 400 nm and at least one particle in a range of greater than 1000 nm for every 1, 2, 5, or 10 particles at any of 500, 600, 700, 800, or 900 nm.

Still referring to FIG. 33, as illustrated, a first mixing technique/type yields a first range of particle sizes 3312, such as where the first range of particle sizes has a first peak distribution of particle sizes, such as at about 1500 nm or in a range greater than 900 nm. Generally, as the energy of impacts and/or applied shear forces increases, the resulting particle sizes of the oil droplets/micelles/cells in an aqueous solution decrease. Thus, the second mixing technique 3314 delivering more energy per unit mixing time at a given concentration yields a distribution of smaller particle sizes 3314 than the larger particles sizes 3312 of the first mixing technique/type, such as a decrease in a mean particle size of greater than 50, 100, 200, 300, or 400 nm relative to the first mixing technique. As illustrated, the second range of particle sizes has a second peak distribution of particle sizes, such as at about 700 nm or in a range less than 900 nm. Similarly, a third mixing technique/type delivers still more energy per unit volume to an emulsion, which further reduces the mean particle size 3316, such as by an additional amount greater than 50, 100, 200, 300, or 400 nm relative to the second mixing technique or greater than 200, 400, or 600 nm relative to the first mixing technique.

While there is overlap in generated particle sizes/oil droplet sizes in emulsions prepared with different techniques, generally particle sizes get smaller in the order of mixing by/with: hand, a hand held low speed mixer (<5,000 rpm), a high speed mixer (>5000 rpm), a rotor-stator mixer, an ultrasonic mixer, and a high shear/impact mixer. An increased mixing time for a given mixer type reduces mean particle size of the particles, but to a limit, where the minimum mean particle sizes are smaller for a professional: high shear mixer and then larger for an ultrasonic mixer, still larger for a rotator stator mixer, and still larger for a hand-held kitchen mixer. Generally, the higher the shear strength and if applicable an impact force of a mixer, the easier it is to blend liquids of differing viscosities and/or densities.

Referring now to FIG. 4, an emulsion preparation system 3400 is described. Generally, two or more components 620 are mixed in an emulsion, such as a hydrophilic component, such as an oil, and a hydrophilic component, such as an aqueous solution and/or water. Optionally and preferably three or more components are mixed in an emulsion, such as water 642, an oil, and an emulsifier 650. Examples of an oil include THC, an initial THC concentrate 634, and/or an emulsion oil, such as canola oil, coconut oil, a medium chain triglyceride, a medium chain oil, a short chain triglyceride, a short chain oil, and/or a fat, such as a long-chain triglyceride. Optionally, a solvent 640, such as ethanol, is used in a mixture, a solution, and/or the emulsion. Optionally, one or more additives, referred to here as an emulsion additive 649, are added to the emulsion during formation and/or after formation. Optional emulsion additives 649 include any of the nootropics 220 described supra.

Still referring to FIG. 4, the components 620 are mixed in a mixer 3410, such as in a first mixer type 3412, in parallel and/or optionally and preferably later in a second mixer type 3414, and/or in parallel and/or optionally and preferably later in a third mixer type 3416, where the first, second, and third mixer types are optionally n mixer types where n is a positive integer greater than 1, 2, 3, 4, or 5. Optionally and preferably, the second mixer type 3414 delivers more energy per unit volume per unit time than the first mixer type 3412 and less energy per unit volume per unit time than the third mixer type 3416.

Still referring to FIG. 4, the optional step of dilution 3430 is important and even critical in certain situations. For example, emulsifying an aqueous product, such as a drink, with THC and an emulsifier and later carbonating the beverage is extremely time inefficient and energy wasting versus emulsifying a concentrated oil/water/emulsifier mixture and later diluting, such as a dilution with a drink/aqueous product of greater than 1:2; 2:1, 3:1, 5:1, 10:1, 25:1, 50:1, or 100:1 (aqueous product by mass or volume:oil emulsion by mass or volume) as the mixer delivers emulsifying energy per unit volume. Hence, use of a mixer on an aqueous product already diluted by 100:1 over an emulsion concentrate requires 100× (actually more due to remixing time in a container) that of mixing the emulsion and thus 100× the energy. Thus, if a THC/emulsifier/water mix of at least 1 gram of THC per 100 mL of solution is mixed for at least 10 minutes, such as at a mixing temperature exceeding 50° C., the mixing time for the same emulsion diluted 100:1 is 1000+ minutes, which is energy and time inefficient. Hence, an optional and preferred embodiment of forming an aqueous product containing droplets/micelles/cells or THC, such as in an emulsifier, in water is to make a concentrate of the emulsion, such as at a concentration exceeding 1, 2, 3, 5, 10, 20, 50, 100, or 200 grams/liter and then diluting the concentrated emulsion as a step in product formation 3440 of an aqueous product, such as a soda and/or a THC emulsion additive. The name nano-fuel is herein dubbed an example of a THC emulsion additive.

Still referring to FIG. 4, the optional step of temperature control 3420, such as with a temperature controller is used to maintain a temperature of a mixing vessel and/or the components 620 in the mixing vessel to a temperature exceeding 40, 50, or 60° C. while mixing for a time period exceeding 1, 2, 5, 10, 30, or 60 minutes and preferably in a range of 55-90° C., 60-80° C., and/or 65-75° C. for the aforementioned time period, which facilitates emulsion formation by reducing viscosity of the THC concentrate 634, such as to a viscosity of less than 500, 250, 100, 50, 20, or 10 newton-second per square meter (N-sec/m²) or Pascal-second (Pa-sec).

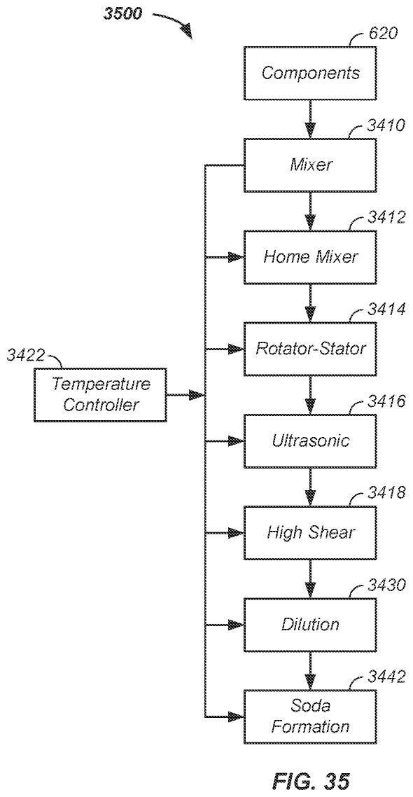

Referring now to FIG. 35, an example of an order of use of mixers to make a THC emulsion used in formation of a product is provided. As illustrated, components are mixed with a mixer 3410, such as a home/kitchen mixer 3412, a rotator-stator mixer 3414, an ultrasonic mixer 3416, and/or a high shear mixer 3418, such as a high shear/impact mixer described infra, in any order followed by an optional dilution 3430 step, such as with water and/or an aqueous product, and/or a hydrophilic product, such as into a final product and/or a product to be further amended, such as in a soda formation step 3442, as described supra. Optionally a temperature controller 3422 is used to control temperature, such as control of a mixing blade, mixing container, and/or emulsion components, as described supra. The temperature controller optionally heats the THC to enhance mixing prior to application of shear forces and/or cools the resulting emulsion, after heating in a mixing step, to reduce loss and/or degradation of THC in the emulsion, such as by phase changing the THC into a gas phase.

Still referring to FIG. 35, in one case the components 620 are mixed in a rotator-stator mixer 3414 to form an initial emulsion with an initial mean particle size (excluding particles under 20, 50, 100, or 200 nm in diameter), such as a mean particle size in a range of any of 400 to 2500 nm, 700 to 2500 nm, 1000 to 2500 nm, 400 to 2000 nm, 700 to 2000 nm, and 1000 to 2000 nm. Optionally and preferably, the THC emulsion is at a concentration of greater than 100, 200, 250, 500, or 1000 mg THC/10, 20, 30, 50, 100, 250, 500, or 1000 mL total volume. In this case, the initial emulsion is optionally diluted in a step of forming the final product and/or is further mixed to form a secondary emulsion in a secondary mixing step using the ultrasonic mixer 3416 and/or the high shear mixer 3418. In a sub-case where the ultrasonic mixer is used to form the secondary mixer, substantially all or part of the secondary emulsion is optionally mixed to form a tertiary emulsion using the high shear mixer 3418. In this case, the initial emulsion, the secondary emulsion, and the tertiary emulsion are optionally mixed in any ratio to form the long/time release emulsion 3320. Optionally, the number of passes through a mixer, such as through the high-shear mixer 3418 is a positive integer, such as 1, 2, 3, 4, 5, 6, 7, or more.

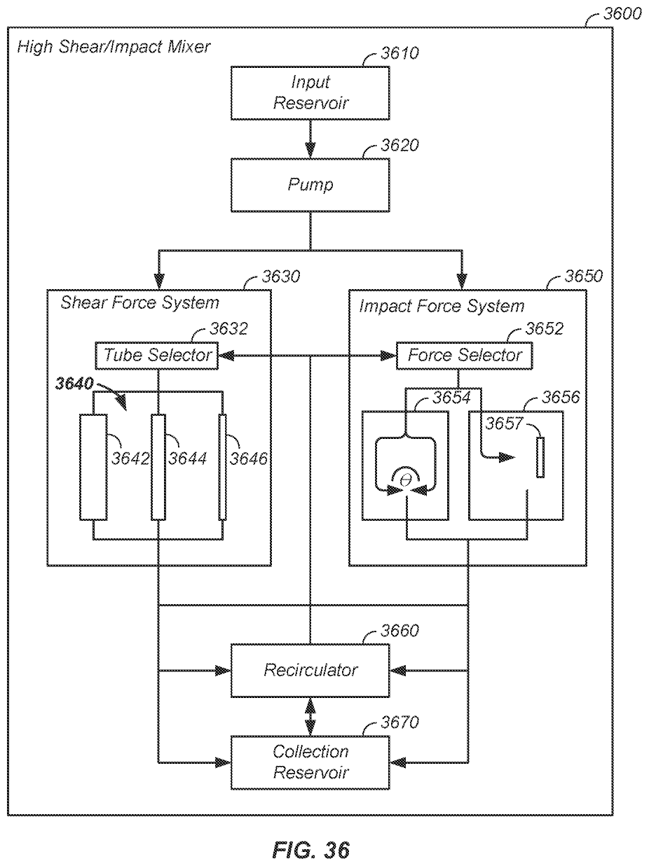

Referring now to FIG. 36, a high shear/impact mixer 3600 is described, which is optionally a high shear mixer and/or a high impact mixer. A high shear mixer provides a shear force to a particle, such as an oil droplet in water, that disrupts the particle to form smaller particles. Optionally and preferably, the shear force is a shear field and/or a uniform shear field. Typically, a high shear force is achieved by forcing a fluid, such as water, and emulsifier, and oil, through a tube that is substantially longer than it is wide, as further described infra. A representative example of a high shear mixer is a shear force system 3630, as illustrated. A high impact mixer, such as illustrated as an impact force system 3650, forces the fluid, such as the water, the emulsifier, and the oil, into a zone opposed by its own pressure and/or against a solid surface, which breaks apart particles, such as cells and/or micelles into smaller particles.

Still referring to FIG. 36, an example of a high shear/impact mixer 3600 is provided. An input reservoir 3610 holds the components 620, which are optionally and preferably pre-emulsified, such as with the home mixer 3412, the rotator-stator mixer 3414, and/or the ultrasonic mixer 3416. One or more pumps 3620 push/pull the pre-emulsion/components into one or both of the shear force system 3630 and/or the impact force system 3650, which are optionally configured separately, in series, and/or in parallel. In one case, the particles, such as water, an emulsifier, and an oil, such as THC, are initially passed through a shear force system for n cycles, where n is greater than 0, 1, 2, 3, 4, or 5, and then passed through the impact force system 3650 for n cycles, where n is greater than 0, 1, 2, 3, 4, or 5. In another case, after each pass through the shear force system 3630, the particles are passed through the impact force system 3650. Optionally, any first fraction of the particles are recirculated through a given system after each pass and any second fraction of the particles are collected into a collection reservoir after each pass, where the fractions recycled and/or collected are within 1/10^{th} of 1/10^{th}, 2/10^{th}, 3/10^{th}, 4/10^{th}, 5/10^{th}, 6/10^{th}, 7/10^{th}, 8/10^{th}, or 9/10^{th} a total amount of a processed fluid. By collecting fractions of the total fluid processed in differing manners, differing ranges of particle sizes are collected, such as the first range of particle sizes 3312, the second range of particle sizes 3314, and/or the third range of particle sizes 3316, where any number of ranges of particles sizes are optionally collected and/or combined to form the long/time release emulsion 3320. By changing the fractions, the long/time release emulsion 3320 is optionally created to have an initial uptake of a larger fraction of smaller particles, such as a first set of particles at a first total mg level of THC and a second set of particles with a second total mg of THC level, where the first set of particles have a mean particle size at least 100, 200, 300, or 400 nm smaller than the second set of particles and/or the first set of particles contain at least 2, 3, 4, or 5 times the amount of THC of the second set of particles.

Still referring to FIG. 36, the shear force system 3630 is further described. The shear force system 3630 is optionally configured with different tubes 3640 at different times and/or is configured with multiple tubes at the same time and an optional tube selector 3632 mechanically and/or electrically switches a flow path to differing tubes at differing times, such as under control of a manual valve and/or under control of a computer controlled valve/switching system. For example, at a first time the components 620 and/or emulsion is passed through a first tube 3642 comprising a first length and first diameter; at a second time the components 620 and/or emulsion is passed through a second tube comprising a second length and a second diameter; and at a third time the components 620 and/or emulsion is passed through a third tube comprising a third length and a third diameter, where any number of tubes with any internal cross-sectional geometry are used at any number of lengths and/or any mean cross-sectional diameters. The tubes 3640 are optionally configured in series. Optionally and preferably, at a first time a larger diameter tube is used, then after making smaller particles with the first tube, the emulsion is passed through a second tube of smaller diameter and then after still smaller particles are generated, the emulsion is passed through the yet still smaller diameter tube to generate yet still smaller particles. The diameters of the tubes are optionally greater than 1, 2, 5, 10, 50, 100, 1,000, 5,000, 10,000, or 100,000 μpm and are optionally less than 10, 5, 2, 1, 0.5, 0.1, or 0.01 mm. As described, supra, after each pass, any percentage, such as greater than 1, 2, 5, 10, 25, 50, 75, 80, 90, 95, 98, or 99 percent of the volume is optionally collected in the collection reservoir 3670, recirculated through one or more tubes 3640 of the shear force system 3630, and/or is passed through the impact force system 3650, which results in optionally still smaller particles and/or a range of particles.

Still referring to FIG. 36, the impact force system 3650 is further described. In the impact force system 3650, an input set of components 620 and/or an input emulsion is subjected to an impact, such as in an opposing force system 3654, such as fluid being pushed against itself in opposing streams, and/or against an opposing object in an opposing object system 3656, such as against a wall. A force selector system 3652 operates like the tube selector system 3632 to select a fraction of the emulsion, 0 to 100%, to be passed at a given time through the opposing force system 3654 and/or the opposing object system 3656. In the opposing force system 3654, the emulsion is split into two or more paths and then directed toward a flow path from an opposite direction, such as illustrated at angle theta, Θ. Optionally, the angle theta is less than 180, 179, 178, 175, 170, 150, 120, 90, 60, or 30 degrees. In the opposing object system, the solid object 3657 is optionally steel or any material with a hardness rating of greater than 5, 6, 7, 8, or 9 Mohs. As described, supra, after each pass, any percentage, such as greater than 1, 2, 5, 10, 25, 50, 75, 80, 90, 95, 98, or 99 percent of the volume is optionally collected in the collection reservoir 3670, recirculated through one or more tubes 3640 of the shear force system 3630, and/or is passed through the impact force system 3650, which results in optionally still smaller particles and/or a range of particles.

Referring again to FIGS. 32-36, the emulsion is optionally processed until the mean particle size, optionally excluding particle sizes of less than 200, 100, 50, or 20 nm, is less than 1000, 800, 600, 400, or 300 nm. Optionally, and preferably, the transmission of a red, green, or blue laser or any wavelength of light from 200 to 700 nm is greater than 95, 90, 80, 70, 60, or 50% through a 1 millimeter wide sample holder.

Referring again to FIGS. 32-36, output of mixer types, such as the home/kitchen mixer 3412, the rotator-stator mixer 3414, the ultrasonic mixer 3416, and/or the high shear mixer 3418, is combined to form a wide range of particle sizes, such as in a long/time release emulsion 3320. An emulsion prepared with a single mixing technique has exactly one peak concentration as a function of particle size. An emulsion prepared using 2, 3, 4, 5, 6 or more mixing techniques, as described herein, results in an emulsion with more than 1, 2, 3, 4, 5, 6, 7, 8, 9, 10, 12, or 15 peaks in terms of number of particles as a function of particle size, which is key in producing a long/time release emulsion 3320.

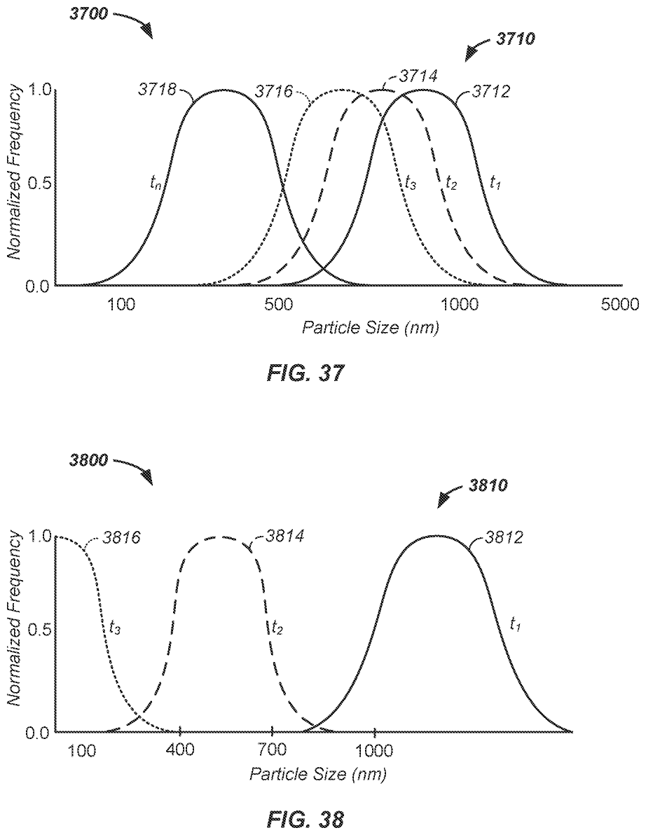

Referring now to FIG. 37, a first method of creating multiple ranges/distributions of particle sizes 3700 is described. As illustrated, a single mixer type, such as the rotator-stator mixer 3414, the ultrasonic mixer 3416, and/or the high-shear mixer 3418, is used, where the THC emulsion is passed through the mixer multiple times to create different particle sizes 3710, such as used in the long/time release emulsion 3320. For instance, as illustrated, the THC emulsion 530 is passed through the high-shear mixer 3418 at a first time, t₁, resulting in a first distribution of particle sizes 3712. Optionally, all or a portion of the THC emulsion 530, now having the first distribution of particle sizes 3712, is passed through the high-shear mixer 3418 at a second time, t₂, resulting in a second distribution of particle sizes 3714. Generally, the additional pass of the THC emulsion 530 through the high-shear mixer 3418 results in a new distribution of particle sizes with a second mean particle size at least 1, 2, 5, or 10 percent smaller than a first mean particle size of the first distribution of particle sizes 3712. For example, if fifty percent of the THC emulsion 530 having the first particle sizes is reprocessed with the high-shear mixer 3418 and then the resulting second distribution of particle sizes 3714 is mixed with the first distribution of particle sizes 3712, then a wider distribution of particle sizes results, such as having two Gaussian peaks with two local maxima, which is an example of a longer term/longer release long/time release emulsion 3320. As illustrated, if a portion of the second distribution of particles sizes 3714 is passed through the high-shear mixer 3418 a third time, t₃, then a third distribution of particle sizes 3716 results. Optionally, the process is repeated n times, tₙ, to yield an n^{th} distribution of particle sizes 3718, where n is a positive integer greater than 1, 2, 3, 4, or 5. By controlling the amount of each batch in the long/time release emulsion 3320 a profile of particle sizes results with n peaks, where each unconvolved peak is of any size relative to the other unconvolved peaks and/or where the particle size distribution is greater than 10, 20, 30, 40, 50, 75, or 100 percent the width, such as full width at half height, of the first distribution of particle sizes 3712. Optionally and preferably at least 25, 50, 75, 90, 95, or 100% of the particles in the THC emulsion 530, such as particles greater than 20 nm, are in a range of 100 to 400 nm, 100 to 700 nm, 400 to 700 nm, or greater than 700 nm. Optionally and preferably at least 25, 50, 75, 90, 95, or 100% of the particles in the THC emulsion 530, such as particles greater than 20 nm, are greater in size than 100, 200, 300, 400, 500, 600, or 700 nm. Optionally and preferably at least 25, 50, 75, 90, 95, or 100% of the particles in the THC emulsion 530, such as those particles greater than 20 nm, are smaller in size than 2000, 1000, 700, 600, 500, or 400 nm in diameter and/or largest cross-sectional dimension. Optionally and preferably at least 2, 5, 10, 25, or 50% of the particles in the THC emulsion 530, such as particles greater than 20 nm, are smaller in size than 700, 600, 500, or 400 nm and at least 2, 5, 10, 25, or 50% of the particles in the THC emulsion 530, such as particles greater than 20 nm, are greater than 700, 800, 900, 1000, or 1500 nm in diameter.

Still referring to FIG. 37, the mixer type, such as the rotator-stator mixer 3414, the ultrasonic mixer 3416, and/or the high-shear mixer 3418, is optionally changed on successive passes of the THC emulsion 530 through the mixer to yield a range of particle sizes.

Referring now to FIG. 38, a second method of creating multiple ranges/distributions of particle sizes 3800 is described, which is optionally used with or without the first method of creating multiple ranges/distributions of particle sizes 3700. Generally, the second method uses the multiple cycling process of the first method, but in place of passing through an identical mixer, such as for a fixed period of time and/or for different mixing times, the THC emulsion 530 is passed through a series of tubes, optionally and preferably of decreasing diameter size with time, to create a second set of different particle sizes 3810, such as used in the long/time release emulsion 3320. For instance, as illustrated, the THC emulsion 530 is passed through the high-shear mixer 3418, such as through the first tube 3642 at first time, t₁, resulting in a first range of particle sizes 3812. Optionally, all or a portion of the THC emulsion 530, now having the first range of particle sizes 3812, is passed through the high-shear mixer 3418 configured with the second tube 3644 at a second time, t₂, resulting in a second distribution of particle sizes 3714. The tubes are optionally manually changed between passes, are in series, are in parallel, and/or are selected, such as with a manual or computer controlled valving mechanism. Generally, the additional pass of the THC emulsion 530 through the high-shear mixer 3418 results in a new range of particle sizes with a second mean particle size at least 1, 2, 5, 10, 20, or 50 percent smaller than a first mean particle size of the first range of particle sizes 3812. For example, if 20, 40, or 60 percent of the THC emulsion 530 having the first particle sizes is reprocessed with the high-shear mixer 3418 and then the resulting second range of particle sizes 3814 is mixed with the first distribution of particle sizes 3812, then a wider distribution of particle sizes results, such as having two Gaussian peaks with two local maxima that are roughly proportionally sized to the percent treated with each process, which is an example of a longer term/longer release long/time release emulsion 3320. The process is optionally repeated n times, such as the illustrated third time using the third tube 3646 to yield a third range of particle sizes 3816. Optionally an output distance between a tube and the solid object 3657 is reduced with successive passes to yield similar decreases in mean or median particle sizes. Generally, additional passes of the THC emulsion 530 through the high-shear mixer 3418 with decreasing tube diameters and/or cross-sectional areas and/or distances to the solid object 3657 results in new ranges of particle sizes with a second mean particle size at least 1, 2, 3, 5, 10, or 20 percent smaller than a first mean particle size of the first range of particle sizes 3712. For example, if fifty percent of the THC emulsion 530 having the first particle sizes is reprocessed with the high-shear mixer 3418 and then the resulting second distribution of particle sizes 3714 is mixed with the first distribution of particle sizes 3712, then a wider distribution of particle sizes results, such as having two Gaussian peaks with two local maxima, which is an example of a longer term/longer release long/time release emulsion 3320. Similarly, the mixing time is increased in a mixer, the frequency of an ultrasonic mixer is increased, and/or a gap between the rotor and the stator is decreased to yield a range of particle sizes.

Still yet another embodiment includes any combination and/or permutation of any of the elements described herein.

Herein, a set of fixed numbers, such as 1, 2, 3, 4, 5, 10, or 20 optionally means at least any number in the set of fixed number and/or less than any number in the set of fixed numbers.

Herein, any number optionally includes a range of numbers such as the number, n,±1, 2, 3, 4, 5, 10, 20, 25, 50, or 100% of that number.

The particular implementations shown and described are illustrative of the invention and its best mode and are not intended to otherwise limit the scope of the present invention in any way. Indeed, for the sake of brevity, conventional manufacturing, connection, preparation, and other functional aspects of the system may not be described in detail. Furthermore, the connecting lines shown in the various figures are intended to represent exemplary functional relationships and/or physical couplings between the various elements. Many alternative or additional functional relationships or physical connections may be present in a practical system.

In the foregoing description, the invention has been described with reference to specific exemplary embodiments; however, it will be appreciated that various modifications and changes may be made without departing from the scope of the present invention as set forth herein. The description and figures are to be regarded in an illustrative manner, rather than a restrictive one and all such modifications are intended to be included within the scope of the present invention. Accordingly, the scope of the invention should be determined by the generic embodiments described herein and their legal equivalents rather than by merely the specific examples described above. For example, the steps recited in any method or process embodiment may be executed in any order and are not limited to the explicit order presented in the specific examples. Additionally, the components and/or elements recited in any apparatus embodiment may be assembled or otherwise operationally configured in a variety of permutations to produce substantially the same result as the present invention and are accordingly not limited to the specific configuration recited in the specific examples.

Benefits, other advantages and solutions to problems have been described above with regard to particular embodiments; however, any benefit, advantage, solution to problems or any element that may cause any particular benefit, advantage or solution to occur or to become more pronounced are not to be construed as critical, required or essential features or components.

As used herein, the terms “comprises”, “comprising”, or any variation thereof, are intended to reference a non-exclusive inclusion, such that a process, method, article, composition or apparatus that comprises a list of elements does not include only those elements recited, but may also include other elements not expressly listed or inherent to such process, method, article, composition or apparatus. Other combinations and/or modifications of the above-described structures, arrangements, applications, proportions, elements, materials or components used in the practice of the present invention, in addition to those not specifically recited, may be varied or otherwise particularly adapted to specific environments, manufacturing specifications, design parameters or other operating requirements without departing from the general principles of the same.

Although the invention has been described herein with reference to certain preferred embodiments, one skilled in the art will readily appreciate that other applications may be substituted for those set forth herein without departing from the spirit and scope of the present invention. Accordingly, the invention should only be limited by the Claims included below.

1. A method for producing a Cannabis product, comprising the steps of:
combining tetrahydrocannabinol and ethanol to form a THC-alcohol compound, said THC-alcohol compound comprising less than fifty percent water by mass; and
subjecting the THC-alcohol compound to a shear force in excess of 100 sec⁻¹.

2. The method of claim 1, further comprising the step of:
passing the THC-alcohol compound through an opening of less than five millimeters in diameter, said step of passing resultant in the shear force exceeding 100,000 sec⁻¹.

3. The method of claim 2, further comprising the step of:
infusing, after said step of passing, an edible with the THC-alcohol compound, wherein a ratio of the ethanol to the tetrahydrocannabinol exceeds 1:2 by mass.

4. The method of claim 3, further comprising the step of:
adding a nootropic to said THC-alcohol compound prior to said step of infusing.

5. The method of claim 2, further comprising the step of:
infusing, after said step of passing, a preroll of Cannabis flower with the THC-alcohol compound.

6. The method of claim 5, further comprising the step of:
removing, after said step of subjecting, at least ten percent of the ethanol from the THC-alcohol compound.

2. hod of claim 2, further comprising the step of:
repeating said step of passing, after in said step of passing, passing at least twenty percent of the THC-alcohol compound through said opening.

8. The method of claim 2, further comprising the step of:
adding a nootropic to said tetrahydrocannabinol.

9. The method of claim 1, further comprising the step of:
including a psychoactive agent in said THC-alcohol compound.

10. The method of claim 2, further comprising the steps of:
in a first process, decreasing viscosity of the tetrahydrocannabinol by at least 2000 centipoise, in the formation of an intermediate tetrahydrocannabinol compound, via addition of at said ethanol to said tetrahydrocannabinol; and
in a subsequent process, increasing viscosity of the intermediate tetrahydrocannabinol compound by at least 2000 centipoise by combining the intermediate tetrahydrocannabinol compound into at least one of:
an edible; and
a preroll of Cannabis flower.

11. The method of claim 2, further comprising the steps of:
decreasing viscosity of the tetrahydrocannabinol from a viscosity in excess of 2000 centipoise to a viscosity less than 500 centipoise through addition of said ethanol to said tetrahydrocannabinol to form said THC-alcohol compound; and
further decreasing viscosity of the THC-alcohol compound to less than 100 centipoise through addition of a least one part water to one part of the THC-alcohol compound in formation of at least one of:
a beverage;
a tincture; and
a vape pen solution.

12. The method of claim 11, further comprising the step of:
labeling a container holding the beverage with a QR-code, said QR-code linking to at least one of:
a location of harvest of a plant containing the tetrahydrocannabinol in said beverage container;
a date of harvest of Cannabis containing the tetrahydrocannabinol in said beverage container;
a THC strain of the tetrahydrocannabinol in said beverage container;
a method of extraction of the tetrahydrocannabinol in said beverage container;
a laboratory report containing at least heavy metal impurities in said beverage container;
a laboratory report listing agrochemicals in said beverage container; and
a distribution chain of the tetrahydrocannabinol in said beverage container.

13. The method of claim 2, further comprising the step of:
adding, after said step of passing, the THC-alcohol compound to a vape pen solution.

14. The method of claim 1, further comprising the step of:
combining the THC-alcohol compound with a commercially available product to yield a THC version of the commercially available product.

15. The method of claim 1, further comprising the step of:
combining the THC-alcohol compound with a sweetener to yield a THC product.

16. The method of claim 1, further comprising the step of:
diluting said THC-alcohol compound by a factor of greater than 10:1 in formation of a THC product.

17. The method of claim 1, further comprising the step of:
encapsulating the THC-alcohol compound in at least one of:
a dissolvable capsule;
a polymer; and
a tablet.

18. An apparatus, comprising:
at least one of an edible and a preroll of Cannabis flower infused with a THC-alcohol compound, said THC-alcohol compound comprising less than fifty percent water by mass, said THC-alcohol compound subjected to a shear force in excess of 100 sec⁻¹.

19. The apparatus of claim 18, said THC-alcohol compound encapsulated in at least one of:
a dissolvable capsule;
a polymer; and
a tablet.

20. The apparatus of claim 18, said THC-alcohol compound comprising particles, said particles comprising a mean largest cross-sectional length of less than 400 nm.
